(19) United States
(12) Patent Application Publication
(10) Pub. No.: US 2026/0079151 A1
(43) Pub. Date: Mar. 19, 2026

(54) THREE DIMENSIONAL IMMUNOMECHANICAL ORGANOID MODEL

(71) Applicant: UNIVERSITY OF NOTRE DAME DU LAC, South Bend, IN (US)

(72) Inventors: Alice BURCHETT, South Bend, IN (US); Meenal DATTA, South Bend, IN (US)

(73) Assignee: UNIVERSITY OF NOTRE DAME DU LAC, South Bend, IN (US)

(21) Appl. No.: 19/332,968

(22) Filed: Sep. 18, 2025

Related U.S. Application Data
(60) Provisional application No. 63/696,022, filed on Sep. 18, 2024.

(51) Int. Cl. G01N 33/50; C12N 5/0786

(52) U.S. Cl. CPC G01N 33/5055; C12N 5/0645; C12N 2513/00

(57) ABSTRACT

A 3D immunomechanical model of myeloid cells for use in basic research and drug screening that allows for observation of the myeloid-mechanical interaction in the context of a range of different mechanical and immunological-related diseases. The model is also suitable for applications such as high-throughput drug screening and experiments in a microgravity environment such as low earth orbit.

### RELATED APPLICATIONS

The present application claims priority under 35 U.S. C. § 119(e) to U.S. Provisional Ser. No. 63/696,022 filed Sep. 18, 2024, the entire disclosure of which is hereby incorporated by reference in its entirety.

### STATEMENT REGARDING FEDERAL FUNDING

This invention was made with government support under grants K22 CA258410, R35 GM151041 and TR002529 awarded by the National Institutes of Health (NIH) and grant CMMI2425684 awarded by the National Science Foundation (NSF). The government has certain rights in the invention.

### BACKGROUND OF THE INVENTION

From atherosclerotic plaques to tuberculosis granulomas to solid malignant tumors, macrophages play important roles in immune effector function and orchestration in aberrant tissue masses, and as active constituents of the mechanical microenvironment. As innate immune cells, they are not only part of the first line of defense against pathogens, but they also contribute to tissue repair and help coordinate the broader immune response. Macrophage phenotype is highly plastic and ebbs between pro- and anti-inflammatory states, as these cells sense and correspondingly respond to diverse and dynamic microenvironments. Macrophages present in tissues are either resident or derived from circulating monocytes that differentiate into macrophages upon vascular extravasation and tissue infiltration. During an inflammatory response, an injured or diseased site will accumulate macrophages, both through the recruitment of circulating monocytes and local proliferation of bone marrow and embryonic-derived macrophages. For example, upon tissue injury, vascular endothelial cells upregulate adhesion molecules that allow patrolling monocytes to adhere to the vessel wall, where they withstand shear stress from blood flow, and eventually enter the tissue between endothelial junctions.

While macrophage phenotype and function, known also as polarization, is traditionally thought to exist as either pro-inflammatory (“M1-like”) or anti-inflammatory (“M2-like”), their dynamic cell state can lie on a continuum between inflammation-accelerating and inflammation-inhibiting responses. Macrophages adopt and shift between these polarizations in any tissue; generally, equilibrium between the two ends of the spectrum is essential for homeostatic tissue maintenance and repair. However, an over-or under-active macrophage response can disrupt this tenuous balance, particularly in cases where macrophages accumulate in large quantities. Thus, there is a need for a physiologically relevant in vitro model is essential to understanding how macrophages respond—and contribute—to mechanical stimuli in the body. The present invention satisfies this need.

### SUMMARY OF THE INVENTION

Elevated macrophage populations in diseased tissue are often correlated with worse prognosis, particularly in diseases where altered tissue mechanics contribute to the pathophysiology. Cancer, wound healing, bacterial infections, and other disease settings involve altered tissue mechanics, which impact immune surveillance and response. For example, atherosclerotic plaques physically disrupt blood flow, and their mechanical stability determines if they will rupture, leading to downstream ischemic events. Macrophages accumulate in plaques, where they contribute to this mechanical instability, increasing the risk of life-threatening events such as a stroke. Macrophages infiltrate tumor microenvironments in high numbers, with the density of tumor-associated macrophages correlating with worse prognoses in cancers including glioblastoma, ovarian cancer, and breast cancer. In glioblastoma, macrophages can comprise up to 50% of the tumor bulk, promoting tumor progression and treatment resistance. Macrophages also contribute to increased collagen deposition in hypertrophic scars and heart attack scarring, leading to host tissue damage and diminished function.

In addition to classical biochemical cues, macrophages also respond to mechanical stimuli such as shear stress, tissue viscoelasticity, cyclic compression or stretching, and hydrodynamic pressure changes. This response has been characterized previously in the context of the cardiovascular and skeletomuscular systems. Macrophages experience a wide range of tissue mechanical properties, with Young's moduli on the order of single kilopascals in the brain to tens of gigapascals in bone. In vitro experiments show that macrophages have a stronger inflammatory response when cultured on stiffer 2-D substrates. However, the opposite effect is observed when cells are cultured in a 3-D matrix. Macrophages in stiffer matrices in vitro and in vivo have a more immunosuppressive, M2-like phenotype.

The present disclosure characterizes external stimuli, such as solid stress that macrophages generate through 3-D growth in a confining agarose gel, simulating the mechanics of the tissue microenvironment independent of confounding biochemical cues or matrix degradation. For example, macrophages (RAW264.7) were embedded in agarose gels of varying substrate concentrations to span a range of physiologically relevant stiffnesses. As the agarose-embedded cells proliferated to form spheroids, they displaced the surrounding gel, similar to physiological macrophages en masse (e.g., in plaques, traumatic brain injuries, granulomas, cancerous tumors) generating and exerting growth-induced solid stress on the surrounding tissue (Stylianopoulos et al., Proc Natl Acad Sci U S A 109:15101-15108.doi.org/10.1073/pnas.1213353109). Spheroids in softer gels grew to much larger sizes and caused larger total displacements of the surrounding gel compared to spheroids in stiffer gels. Pro-inflammatory stimulation with LPS also led to an increase in spheroid size, though this effect was reversed with the addition of interferon-γ (IFNγ). The mechanosensitive ion channels Piezo1 and transient receptor potential vanilloid 4 (TRPV4) both decreased in expression with increased stiffness and externally applied compression. Markers of both pro- and anti-inflammatory functional states increased with stiffness and compression. Overall, this work highlights a novel, tunable, and high throughput method of interrogating macrophage immunomechanics and mechano-immunology, with relevance to a wide range of diseases.

Accordingly, certain embodiments of the invention provide three dimensional organoids comprising live myeloid cells, and in particular, live macrophage cells. In some embodiments, the organoids may comprise at least one other population of cells in addition to the myeloid cells.

In some embodiments, the organoid is embedded in a hydrogel comprising agarose and optionally, gelatin. The amount of agarose in the hydrogel may be about 0.1% to about 10% of total weight of the hydrogel, or about 0.5% to about 5% of total weight of the hydrogel. In some embodiments, the hydrogel comprising the organoid comprises a Young's moduli of about 0.5 kPa to about 150 kPa, or about of about 0.5 kPa to about 50 kPa.

Further embodiments of the invention provide methods of testing immunomechanical stress on immune cells comprising: subjecting the organoid to the stimulus; and measuring an immunological and/or physical response of the organoid to the stimulus as compared to an immunological and/or physical response of the organoid prior to contact with the stimulus or in the absence of the stimulus, wherein the stimulus comprises one or more of: a) subjecting the organoid to a compressive force; b) contacting the organoid with one or more compounds of interest; and c) subjecting the organoid to microgravity; and wherein the organoid comprises a first population of live myeloid cells and optionally, a second population of cells that are not myeloid cells, and wherein the organoid is embedded in a hydrogel.

Preferably, the first population of live myeloid cells comprise at least one of a monocyte, granulocyte, erythrocyte, and megakaryocyte, and more preferably, macrophages.

In some embodiments, and organoid may further comprise a second population of cells comprising one or more of adipocytes, blood cells, bone marrow cells, cardiac cells, chondrocytes, dendritic cells, endothelial cells, eosinophils, epithelial cells, fibroblasts, glial cells, glucagon-producing alpha cells, hair cells, hepatocytes, insulin-producing beta cells, keratinocytes, Langerhans cells, B and T lymphocytes, macrophages, mast cells, melanocytes, microvascular cells, monocytes, muscle cells, neurons, neutrophils, pancreatic polypeptide-producing cells, pancreatic ductal cells, pancreatic cells, parathyroid cells, pericytes (Rouget cells), pigment cells, plasma cells, pituitary cells, reticular cells, rod cells, skeletal cells, somatostatin-producing delta cells, stromal cells, and thyroid cells.

In other embodiments, the second population of cells derived from a subject having a disease or condition resulting in an accumulation of macrophages, wherein the disease or condition comprises one or more of atherosclerosis, atherosclerotic plaques, Chronic Obstructive Pulmonary Disease (COPD), Covid-19, Hashimoto's Thyroiditis, Hepatic Fibrosis, hypertrophic scarring, an infectious disease, inflammatory phase of fatty liver disease (NASH), an intestinal disease, a neurodegenerative disease, obesity, psoriasis, rheumatoid arthritis (RA), Systemic Sclerosis systemic lupus erythematosus (SLE), Type I Diabetes, and tumors.

In other embodiments, the immunological and/or physical response comprises one or more of an increase in organoid size, a decrease in organoid size, an increase in a number of cells of the first population of live myeloid cells, a decrease in a number of cells of the population of live myeloid cells, an increase in one or more cellular markers of cells of the population of live myeloid cells, and a decrease in one or more cellular markers of cells of the population of live myeloid cells.

In some embodiments, the immunological and/or physical response comprises one or more of an increase in organoid size, a decrease in organoid size, an increase in a number of cells of the second population of cells, a decrease in a number of cells of the second population of cells, an increase in one or more cellular markers of a cell of the second population of cells, and a decrease in one or more cellular markers of cells of the second population of cells.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C. Macrophage spheroids embedded in agarose form aggregates with sustained viability (A) Percent of live cells one day after embedding in agarose gels of varying concentrations. There was no significant difference between any groups, and each group had n=3. (B) MTT assay results for cells embedded in agarose at varying concentrations at three time points (n=3). (c) Representative images of spheroids at day 5, stained with calcein-AM for live cells, and propidium iodide for dead cells. Scale bar is 200 μm. Representative images at day 16 stained for live and dead cells. Scale bar is 200 μm. Error bars represent SEM and asterisks indicate statistical significance (*p<0.05, ** p<0.01, *** p<0.001, **** p<0.0001).

FIG. 2A-B. Agarose concentration modulates gel stiffness, mechanical interactions, and spheroid expansion (A) Representative images of spheroids stained for actin in varying stiffness gels. (B) Representative COMSOL models of the strain field generated from each spheroid as it expands into a viscoelastic medium.

FIG. 3A-D. Spheroid size varies with agarose stiffness and macrophage stimulation/polarization (A) Representative maximum intensity projections of actin-stained spheroids in 0.5%, 1%, and 2% agarose at day 4. Scale bar is 200 μm. (B) Spheroid area as measured from phase-contrast images for 0.5%, 1%, and 2% agarose. (C) Spheroid area comparison between different agarose concentrations with and without the addition of LPS. (D) Spheroid area comparison between regular 1% agarose cultures, and 1% agarose cultures with the addition of M1-like polarizing agents, M2-like polarizing agents, LPS, and hypoxic culture conditions. Between 26 and 87 spheroids were measured in each condition, error bars represent SEM and asterisks indicate statistical significance (*p<0.05, **p<0.01, ***p<0.001, ***p<0.0001).

FIG. 4A-F. Macrophages show transcriptional response to both mechanical and chemical stimuli qPCR analysis of genes related to proliferation and apoptosis (A-B), mechanosensitive ion channels (C-D), and macrophage polarization (E-F). Asterisks represent the p-value (*p<0.05, *p<0.01, ***p<0.001, ****p<0.0001) from a Mann-Whitney test comparing each condition to the 1% agarose condition (also labeled “Ctrl”), and error bars represent SEM (n=3-6). Bars without an asterisk notation have no significant difference from the 1% control condition.

FIG. 5. Experimental timeline The endpoint assays displayed in the main FIGS. 1-4 were performed at different timepoints, indicated in this timeline

FIG. 6A-B. Mechanical testing of agarose gels (A) Representative stress-strain curves of 2%, 1%, and 0.5% agarose samples. (B) Average Young's moduli of agarose gels at day 1 and day 17 after formation. N=3-4 for each condition.

FIG. 7A-C. Generation of viable PDOs for long-term culture. (A) Organoids generated from a range of different primary glioma lines can incorporate immune cells, such as microglia, and maintain viability for at least four weeks. (B-C) Different combinations of immune cells and primary glioma cells have distinct growth rates in sealed cryovials, as documented via weekly measurements of the spheroid cross-sectional area.

FIG. 8A-C. Optimization of spatial analysis strategies for organoids. (A) Overview of Xenium spatial transcriptomic slide layout to analyze 8 distinct organoid sections on a single slide. (B) Immunofluorescent staining of FFPE-processed organoids grown in space (microgravity) and ground controls. (C) Hematoxylin and Eosin staining of serial sections of these organoids.

### DETAILED DESCRIPTION OF THE INVENTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, PCR amplification, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative”limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5,. 9, 10. It also means 1.0, 1.1, 1.2. 1.3,. 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

As used herein, the terms “increase,” “increases,” “increased,” “increasing,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

Elastic modulus, or modulus of elasticity, refers to the ability of a hydrogel material to resist deformation, or, conversely, an object's tendency to be non-permanently deformed when a force is applied to it. The clastic modulus of an object is defined as the slope of its stress-strain curve in the elastic deformation region: λ=stress/strain, where λ is the elastic modulus in Pascal's; stress is the force causing the deformation divided by the area to which the force is applied; and strain is the ratio of the change caused by the stress to the original state of the object. Specifying how stresses are to be measured, including directions, allows for many types of elastic moduli to be defined. The three primary elastic moduli are tensile modulus, shear modulus, and bulk modulus.

Tensile modulus (E) or Young's modulus refers to an object's response to linear strain, or the tendency of an object to deform along an axis when opposing forces are applied along that axis. It is defined as the ratio of tensile stress to tensile strain. It is often referred to simply as the elastic modulus. The shear modulus or modulus of rigidity refers to an object's tendency to shear (the deformation of shape at constant volume) when acted upon by opposing forces. It is defined as shear stress over shear strain. The shear modulus is part of the derivation of viscosity. The shear modulus is concerned with the deformation of a solid when it experiences a force parallel to one of its surfaces while its opposite face experiences an opposing force (such as friction). The bulk modulus (K) describes volumetric elasticity or an object's resistance to uniform compression, and is the tendency of an object to deform in all directions when uniformly loaded in all directions. It is defined as volumetric stress over volumetric strain, and is the inverse of compressibility. The bulk modulus is an extension of Young's modulus to three dimensions.

Drug dosage forms can be devised by those skilled in the art, as shown for example in Ansel and Popovich, Pharmaceutical Dosage Forms and Drug Delivery Systems, 5th Edition, (Lea & Febiger 1990), Gennaro (Ed.), Remington's Pharmaceutical Sciences, 19th Edition (Mack Publishing Company 1995), and Ranade and Hollinger, Drug Delivery Systems (CRC Pres 1996); and Nair et al., J Basic Clin Pharm. 2016 Mar; 7 (2):27-31.

Aspects of the invention are described in Burchett et al., Cell Mol Bioeng. 2024 Oct. 14; 17(5):329-344, incorporated by reference herein in it entirely.

A “compound of interest” or “target compound” as used herein may be any compound or agent for which a pharmacological or physiological activity is to be determined such as, e.g., for which a pharmacological or physiological activity on a cell or tissue and/or an interaction between two test compounds/agents is to be determined. Compounds of interest may include organic compounds such as, but not limited to, proteins, peptides, nucleic acids, and/or small organic compounds (aliphatic, aromatic, and mixed aliphatic/aromatic compounds). Candidate compounds may be generated by any suitable techniques, including randomly generated by combinatorial techniques, and/or rationally designed based on particular targets. Where a drug interaction is to be studied, two (or more) test compounds may be administered concurrently, and one (or both) may be known compounds, for which the possible combined effect is to be determined. In some embodiments, two or more test compounds may be administered in a manner similar to in vivo administration for a subject (e.g., similar to staged infusion of two or more test compounds), which may be concurrent administration or sequential administration.

### Embodiments of the Invention

The disclosure generally provides for 3-dimensional (3D) organoids comprising live myeloid cells and methods of testing, for example, biomechanical stress and pharmacological agents, on the live myeloid cells embedded in the 3D organoid.

The term “organoid” refers to a composition of live cells, typically in a carrier media, arranged in a three-dimensional or multi-layered configuration (as opposed to a monolayer). An organoid is an artificial, three-dimensional construct created in vitro to mimic or resemble the functionality and/or histological structure of an organ, tissue, or a portion thereof. Suitable carrier media include hydrogels. Exemplary hydrogels include, but are not limited to, those described herein and in U.S. Patent Publication Nos. 2023/0417740 to Skardal et al. and 2019/0345096 to Welker et al., and U.S. Pat. Nos. 11,629,329 and 12,038,432 to Skardal et al., the contents of each of which are incorporated herein by reference in their entirety. An organoid may comprise one or more (e.g., 1, 2, 3, 4, or more) differentiated cell type(s) depending upon the particular tissue and/or organ being modeled or emulated. In some embodiments, cells may be mixed together with an extracellular matrix, or cross-linked matrix, to form the organoid, while in other embodiments cell aggregates such as, e.g., spheroids and/or organoids may be pre-formed and then combined with the extracellular matrix and/or a composition of the present invention.

In some embodiments, polymer(s) of the hydrogel solution are crosslinkable polymers when subjected to a stimulus such as temperature, pH, ions, etc. Advantageously, the hydrogel solution used is biocompatible, in the sense that it is not toxic to cells. Hydrogels permit the diffusion of dissolved gases (and in particular oxygen and/or carbon dioxide), nutrients, and metabolic wastes to allow the survival, proliferation, differentiation, maturation of cells and/or the production of molecules or molecular assemblies of interest and/or the recapitulation of cellular behaviors of interest. The polymers of the hydrogel solution can be of natural or synthetic origin. For example, the hydrogel solution contains one or more polymers among sulfonate-based polymers, such as sodium polystyrene sulfonate, acrylate-based polymers, such as sodium polyacrylate, polyethylene glycol diacrylate, the gelatin methacrylate compound, polysaccharides, and in particular polysaccharides of bacterial origin, such as gellan gum, or of plant origin, such as pectin, agarose, or alginate. In an embodiment, the hydrogel solution comprises agarose. In some embodiments, the hydrogel solution comprises agarose and gelatin. In some embodiments, the hydrogel consists of agarose, gelatin, and one or more of a buffer, water, or cell culture media. In some embodiments, the hydrogel consists of agarose and one or more of a buffer, water, or cell culture media.

In some embodiments, the hydrogel further may comprises biological polymers and fragments thereof including proteins (laminins e.g., laminin 511, 521, 421) , vitronectins, fibronectins and collagens (Collagen I, II, III, IV)), nonsulfated glycosaminoglycans (hyaluronic acid) or sulfated glycosaminoglycans (chondroitin sulfate, dermatan sulfate, keratan sulfate, heparan sulfate), and synthetic polymers containing units derived from biological polymers or reproducing their properties (RGD unit) and small molecules mimicking attachment to a substrate (Rho-A kinase inhibitors such as Y-27632 or thiazovivin), as well as growth factors, such as TGF-beta and/or EGF. In some embodiments, the hydrogel of the organoid may comprise a protein (e.g., an adhesion protein) and/or proteoglycan, optionally a modified protein and/or modified proteoglycan. In some embodiments, the protein and/or proteoglycan may be modified with one or more functional group(s), such as, e.g., modified with a maleimide, that can bind and/or crosslink to thiolated hyaluronic acid, non-thiolated hyaluronic acid, methacrylated collagen, and/or non-methacrylated collagen. In some embodiments, an organoid may be present in a hydrogel comprising fibronectin, heparin, and/or laminin, optionally a modified fibronectin, heparin, and/or laminin (e.g., modified with a maleimide), or other cell adhesion protein(s) and/or or cell adhesion protein peptide derivative(s). In some embodiments, the hydrogel comprises lipopolysaccharides derived from bacteria.

Preferably, a hydrogel may comprise about 0.1% to about 10% agarose, about 1% to about 8% agarose, about 1.5% to about 6.5% agarose, about 2% agarose to about 5% agarose, or about 0.5% agarose, about 1% agarose, about 1.5% agarose, about 2% agarose, about 2.5% agarose, about 3% agarose, about 3.5% agarose, about 4% agarose, about 4.5% agarose, about 5% agarose, about 5.5% agarose, about 6% agarose, about 6.5% agarose, about 7% agarose, about 7.5% agarose, about 8% agarose, about 8.5% agarose, about 9% agarose, about 9.5% agarose, or about 10% agarose. In some embodiments, a hydrogel of the organoid may comprise about 0.5% to about 5% agarose or about 0.5% to about 2% agarose.

In some embodiments, a hydrogel may further comprise gelatin. As used herein, “gelatin” refers to denatured collagen, in which salt bonds and hydrogen bonds between peptide chains of collagen have been cleaved by acid or alkali or enzymatic treatment etc. so that the collagen changes irreversibly into a water-soluble protein. The collagen may be collected from an animal or a plant or a recombinant collagen may be used. For example, a hydrogel of the organoid may comprise about 0.1% to about 10% gelatin, about 1% to about 8% gelatin, about 1.5% to about 6.5% gelatin, about 2% gelatin to about 5% gelatin, or about 0.5% gelatin, about 1% gelatin, about 1.5% gelatin, about 2% gelatin, about 2.5% gelatin, about 3% gelatin, about 3.5% gelatin, about 4% gelatin, about 4.5% gelatin, about 5% gelatin, about 5.5% gelatin, about 6% gelatin, about 6.5% gelatin, about 7% gelatin, about 7.5% gelatin, about 8% gelatin, about 8.5% gelatin, about 9% gelatin, about 9.5% gelatin, or about 10% gelatin. In some embodiments, a hydrogel of the organoid may comprise about 0.5% to about 5% gelatin or about 0.5% to about 2% gelatin.

In some embodiments, a hydrogel may comprise about 0.5% to about 5% agarose and about 0.5% to about 5% gelatin or about 0.5% to about 3% agarose and about 0.5% to about 3% gelatin.

In some embodiments, the hydrogel comprises a Young's moduli of about 0.5 kPa to about 150 kPa, about 1 kPa to about 125 kPa, about 5 kPa to about 100 kPa, about 10 kPa to about 75 kPa, or about 15 kPa to about 50 kPa. In some embodiments, the hydrogel comprises a Young's moduli of about 0.5 kPa to about 50 kPa, about 35kPa to about 70 kPa, about 50 kPa to about 85 kPa, about 75 kPa to about 100 kPa, about 90 kPa to about 115 kPa, about 110 kPa to about 135 kPa, about 120 kPa to about 140 kPa, about 130 kPa to about 150 kPa, about 140 kPa to about 160 kPa, or about 150 kPa to about 175 kPa. In some embodiments, the hydrogel comprises a Young's moduli of about 1 kPa to about 75 kPa, about 5 kPa to about 50 kPa, of about 8 kPa to about 25 kPa.

Preferably, the hydrogel comprises agarose and further includes one or more buffers and/or a cell culture media. Examples of such media include, but are not limited to, Ames'Medium; Basal Medium Eagle (BME), Click's Medium, Dulbecco's Modified Eagle's Medium (DMEM), DMEM/Nutrient Mixture F12 Ham, Fischer's Medium, Minimum Essential Medium Eagle (MEM), Nutrient Mixtures (Ham's), Waymouth Medium, and William's Medium E. Such media may include carrier and transport proteins (e.g., albumin), biological detergents (e.g., to protect cells from shear forces and mechanical injury), biological buffers, growth factors, hormones, hydrolysates, lipids (e.g., cholesterol), lipid carriers, essential and non-essential amino acids, vitamins, sera (e.g., bovine, equine, human, chicken, goat, porcine, rabbit, sheep), serum replacements, antibiotics, antimycotics, and attachment factors. In some embodiments, the buffer used to fabricate the organoid may include tris-acetate-EDTA (TAE) buffer or Tris-borate-EDTA (TBE) buffer. Exemplary cell culture media includes Dulbecco's Modified Eagle Medium (DMEM, Corning, 10-013-CV cell culture media) optionally supplemented with 10% Fetal Bovine serum. The hydrogel also may comprise water.

In some embodiments, the volume of the hydrogel in which an organoid is present and/or deposited may be about 1 μL to about 150 μL, or about 5 μL to about 50 μL, about or about 5 μL, about 10 μL, about 15 μL, about 20 μL, about 25 μL, about 30 μL, about 35 μL, about 40 μL, about 45 μL, about 50 L, about 55 μL, about 60 μL, about 65 μL, about 70 μL, about 75 μL, about 80 μL, about 85 μL, about 90 μL about 95 μL, about 100 μL, about 105 μL, about 110 μL, about 115 μL, about 120 μL, about 125 μL, about 130 μL, about 135 μL, about 140 μL, about 145 μL, or about 150 μL. In other embodiments, the volume of the hydrogel may be about 100 μL to about 1000 μL, about 150 μL to about 800 μL, or about 200 μL to about 600 μL, or about 100 μL, about 200 μL, about 300 μL, about 400 μL, about 500 μL, about 600 μL, about 700μL, about 800 μL, about 900 μL, or about 1000 μL. In still other embodiments, the volume of the hydrogel may be about 400 μL to about 1000 μL, 500 μL to about 900 μL, or 600 μL to about 800 μL.

In some embodiments, an organoid and/or hydrogel of the present invention may be present in a reservoir and/or a plurality of reservoirs (e.g., wells of a well plate). The reservoir(s) may be any suitable reservoir or container that holds the organoid and/or hydrogel. In some embodiments, the reservoir is a well of a well plate such as, but not limited to, a well in a 6-well plate, a 12-well plate, a 24-well plate, a 48 well plate, a 96-well plate, or 384-well plate.

In some embodiments, one or more types of cells may be incorporated into a composition and/or hydrogel in any suitable form, including as unencapsulated cells, or as cells previously encapsulated in spheroids, or pre-formed organoids. Animal tissue cells encapsulated or contained in polymer spheroids can be produced in accordance with known techniques, or in some cases are commercially available.

In some embodiments, an organoid comprises live myeloid cells, such as monocytes (e.g., dendritic cells, macrophages), granulocytes (e.g., eosinophils, mast cells, basophils, neutrophils), erythrocytes, and megakaryocytes (e.g., platelets), and optionally, a second cell population that in not a myeloid cell.

In some embodiment, myeloid cells or other cells are seeded in the hydrogel of the organoid in an amount of about 5,000 cells to 100,000 cells, about 10,000 cells to about 75,000 cells, about 15,000 cells to about 50,000 cells, or about 20,000 cells to about 30,000 cells. In some embodiment, myeloid cells or other cells are seeded in the hydrogel of the organoid in an amount of about 100,000 cells to 500,000 cells, about 150,000 cells to 450,000 cells, or about 200,000 cells to 400,000 cells. In some embodiment, myeloid cells or other cells are seeded in the hydrogel of the organoid in an amount of about 500,000 cells to 1,500,000 cells, about 600,000 cells to 900,000 cells, or about 700,000 cells to 800,000 cells. In other embodiments, the total number of myeloid cells or other cells range from about 1 million to about 5 million, about 5 million to about 10 million, about 25 million, about 50 million, about 70 million, or about 100 million cells.

In some embodiment, myeloid cells are macrophages and are seeded in the hydrogel of the organoid in an amount of about 5,000 cells to 100,000 cells, about 10,000 cells to about 75,000 cells, about 15,000 cells to about 50,000 cells, or about 20,000 cells to about 30,000 cells. In some embodiment, macrophage cells are seeded in the hydrogel of the organoid in an amount of about 100,000 cells to 500,000 cells, about 150,000 cells to 450,000 cells, or about 200,000 cells to 400,000 cells. In some embodiment, macrophage cells are seeded in the hydrogel of the organoid in an amount of about 500,000 cells to 1,500,000 cells, about 600,000 cells to 900,000 cells, or about 700,000 cells to 800,000 cells. In some embodiments, an organoid of the present invention may comprise about 1, 2, or 5 million to about 10, 25, 50, or 100 million cells per mL. In some embodiments, an organoid of the present invention may comprise about 10 million cells per mL or 20 million cells per mL. In other embodiments, the total number of cells range from about 1 million to about 5 million, about 5 million to about 10 million, about 25 million, about 50 million, about 70 million, or about 100 million cells.

In some embodiments, an organoid comprises a population of myeloid cell (e.g., dendritic cells, macrophages), granulocytes (e.g., eosinophils, mast cells, basophils, neutrophils), erythrocytes, and megakaryocytes (e.g., platelets)) and at least one other population of cells. For example, the other population of cell can include those cells arising from the ectoderm, mesoderm, or endoderm germ cell layers. Such cells include, but are not limited to, bone marrow cells, neurons, glial cells (astrocytes and oligodendrocytes), muscle cells (e.g., cardiac, skeletal), chondrocytes, fibroblasts, melanocytes, Langerhans cells, keratinocytes, endothelial cells, epithelial cells, pigment cells (e.g., melanocytes, retinal pigment epithelial (RPE) cells, iris pigment epithelial (IPE) cells), hepatocytes, microvascular cells, pericytes (Rouget cells), blood cells (e.g., erythrocytes), cells of the immune system (e.g., B and T lymphocytes, plasma cells, macrophages/monocytes, dendritic cells, neutrophils, eosinophils, mast cells), thyroid cells, parathyroid cells, pituitary cells, pancreatic cells (e.g., insulin-producing beta cells, glucagon-producing alpha cells, somatostatin-producing delta cells, pancreatic polypeptide-producing cells, pancreatic ductal cells), stromal cells, adipocytes, reticular cells, rod cells, and hair cells. Other examples of cell types that can be included in an organoid include those disclosed by Spier R. E. et al., eds., (2000) The Encyclopedia of Cell Technology, John Wiley & Sons, Inc., and Alberts B. et al., eds., (1994) Molecular Biology of the Cell, 3^{rd} ed., Garland Publishing, Inc., e.g., pages 1188-1189. Preferably, the other cell is not a myeloid cell.

In some embodiments, the organoid further comprises immune cells such as B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor infiltrating (TIL) cells, gene modified immune cells including hybridomas, drug modified immune cells, and derivatives, precursors or progenitors of any of the cell types listed herein.

In some embodiments, the other population of cells comprise stem cells. Stem cells are believed to have immense potential for therapeutic purposes for numerous diseases. Stem cells have been derived from numerous donor sources, including, but not limited to, embryonic, blast, tissue-derived, blood, and cord-blood cells; organ-derived progenitor cells; and bone marrow stromal cells, among others. Such stem cells can be differentiated along numerous pathways to produce virtually any cell type. These cells can be transplanted either before or after differentiation. Hematopoietic stem cells (HSC) have been used for many years and typically used for treatment of hematopoietic cancers (e.g., leukemias and lymphomas), and non-hematopoietic malignancies (cancers in other organs). Other indications include diseases that involve genetic or acquired bone marrow failure, such as aplastic anemia, thalassemia sickle cell anemia, and autoimmune diseases.

In some embodiments, an organoid may comprise myeloid cells (e.g., macrophages) and cells obtained from a subject, such as, for example, a subject undergoing treatment for cancer and/or that has cancer, or the cells are obtained from a subject having a disease or condition involving an accumulation of myeloid cells (e.g., macrophages) such as with tuberculosis and other infectious diseases, Chronic Obstructive Pulmonary Disease, atherosclerosis, Hepatic Fibrosis, atherosclerotic plaques, obesity, Type I Diabetes, psoriasis, tumors, Hashimoto's Thyroiditis, rheumatoid arthritis (RA), Systemic Sclerosis systemic lupus erythematosus (SLE), respiratory disease such as those associate with covid-19, intestinal disease such as inflammatory bowel disease (IBD) and related intestinal conditions, including Crohn's disease (CD) and ulcerative colitis (UC), neurodegenerative diseases such multiple sclerosis and Alzheimer's disease, inflammatory phase of fatty liver disease (NASH), and hypertrophic scarring. The cells obtained from a subject having a disease or other condition involving an accumulation of myeloid cells are the diseased cells (e.g., cells infected with Mycobacterium tuberculosis, cells infected with SARS-CoV-2, fibrotic cells, etc.). In some embodiments, the organoid is exposed to a lipopolysaccharide derived from a bacterial source (either supplementing the cell culture media or incorporated into the hydrogel during formation of the organoid). Typically, the amount of lipopolysaccharide is about 200 ng/mL. An accumulation of myeloid cells. such as macrophages, refers to a condition the number of myeloid cells is greater than a normal baseline or homeostasis, which can be a reference sample (e.g., non-diseased cells) or compared to a subject prior to or without the condition/disease.

Exemplary neurodegenerative disease include, for example, acute disseminated encephalomyelitis, amyotrophic lateral sclerosis (ALS), retinitis pigmentosa, mild cognitive impairment, senile dementia, progressive supranuclear palsy, subcortical dementias, Wilson disease, multiple infarct disease, arteriosclerotic dementia, AIDS associated dementia, cerebellar degeneration, spinocerebellar degeneration syndromes, Friedreich's ataxia, ataxia telangiectasia, epilepsy-related brain damage, spinal cord injury, restless legs syndrome, striatonigral degeneration, cerebral vasculitis, mitochondrial encephalomyopathies, neuronal ceroid lipofuscinosis, spinal muscular atrophies, lysosomal storage disorders with central nervous system involvement, leukodystrophies, urea cycle defect disorders, hepatic encephalopathies, renal encephalopathies, metabolic encephalopathies, porphyria, bacterial meningitis, viral meningitis, meningoencephalitis, prion diseases, poisonings with neurotoxic compounds, Guillain Barre syndrome, chronic inflammatory neuropathies, polymyositis, dermatomyositis, or radiation-induced brain damage. Other neurodegenerative disorders include Alpha-mannosidosis, Cystinosis, Danon disease, Fabry disease, Farber disease, Fucosidosis, Galactosialidosis, Gaucher disease (Type I, Type II, Type III), GM1 gangliosidosis (infantile, juvenile and adult), I-Cell disease (Mucolipidosis II), Infantile Free Sialic Acid Storage Disease, Juvenile Hexosaminidase A Deficiency, Krabbe disease (Infantile and late onset), Lysosomal acid lipase deficiency (early and late), Metachromatic Leukodystrophy, Pseudo-Hurler polydystrophy (Mucolipidosis IIIA), MPSI (Hurler Syndrome), MPS II (Hunter syndrome), Sanfilippo syndrome Type A (MPS III A), Sanfilippo syndrome Type B (MPS III B), Sanfilippo syndrome Type C (MPS III C), Sanfilippo syndrome Type D (MPS III D), Morquio Type A (MPS IVA), Morquio Type B (MPS IVB), MPS IX (Hyaluronidase Deficiency), MPS VI (Maroteaux-Lamy), MPS VII (Sly Syndrome), Mucolipidosis I (Sialidosis), Mucolipidosis IIIC, Mucolipidosis type IV, Multiple sulfatase deficiency, Niemann-Pick Disease, Type A, Niemann-Pick Disease, Type B, Niemann-Pick Disease, Type C, Neuronal Ceroid Lipofuscinoses, Pompe disease, Pycnodysostosis, Sandhoff disease (infantile, juvenile and adult), Schindler disease, Salla disease (Sialic Acid Storage Disease), Tay-Sachs disease, Wolman disease, chronic traumatic encephalopathy, Alzheimer's disease (AD), Parkinson disease (PD), Huntington disease (HD), Frontotemporal dementia (FTD-3 subtype), Charcot-Marie Tooth disease type 2B, Neuronal ceroid lipofuscinoses/Batten disease (NCL), Creutzfeldt-Jakob disease, Autosomal dominant Spastin hereditary spastic paraplegia (ADHSP), Chediak-Higashi syndrome (CHS), and Inclusion body myositis (IBM).

Exemplary infectious diseases include, but are not limited to, Anthrax, Arboviral diseases (diseases caused by viruses spread by mosquitoes, sandflies, ticks, etc.) such as West Nile virus, eastern and western equine encephalitis, Babesiosis, Botulism, Brucellosis, Campylobacteriosis, Chancroid, Chickenpox, Chlamydia, Cholera, Coccidioidomycosis, Coronavirus (COVID-19), Cryptosporidiosis, Cyclosporiasis, Dengue virus infections, Diphtheria, Ehrlichiosis, Foodborne disease outbreak, Giardiasis, Gonorrhea, Haemophilus influenza (invasive disease), Hantavirus pulmonary syndrome, Hemolytic uremic syndrome (post-diarrheal), Hepatitis A, Hepatitis B, Hepatitis C, HIV infection, Influenza-related infant deaths, Invasive pneumococcal disease, Lead (elevated blood level), Legionnaire disease (legionellosis), Leprosy, Leptospirosis, Listeriosis, Lyme disease, Malaria, Measles, Meningitis (meningococcal disease), Mumps, Novel influenza A virus infections, Pertussis, Pesticide-related illnesses and injuries, Plague, Poliomyelitis, Poliovirus infection (nonparalytic), Psittacosis, Q-fever, Rabies (human and animal cases), Rubella (including congenital syndrome), Salmonella paratyphi and typhi infections, Salmonellosis, Severe acute respiratory syndrome-associated coronavirus disease, Shiga toxin-producing Escherichia coli (STEC), Shigellosis, Smallpox, Syphilis (including congenital syphilis), Tetanus, Toxic shock syndrome (other than streptococcal), Trichinellosis, Tuberculosis, Tularemia, Typhoid fever, Vancomycin intermediate Staphylococcus aureus (VISA), Vancomycin resistant Staphylococcus aureus (VRSA), Vibriosis, Viral hemorrhagic fever (including Ebola virus, Lassa virus, among others), Waterborne disease outbreak, Yellow fever, and Zika virus disease and infection (including congenital).

Exemplary intestinal disease includes, but are not limited to, an inflammatory, infectious or cancerous intestinal disease/disorder, such as, e.g., inflammatory bowel disease, Crohn's disease, ulcerative colitis, irritable bowel syndrome, a colonic bacterial infectious disease, or colorectal cancer.

In some embodiments, cells are tumor cells, such as, e.g., patient biopsy-derived tumor cells, and organoids prepared from such cells may be used to screen potentially effective drugs and/or treatments. Any type of tumor cell may be used in an organoid and/or method of the present invention including, but not limited to, intestinal (small intestine, large intestine, colon, vermiform appendix), lung, breast, prostate, skin, bone, brain, liver, pancreatic, uterine, cervical, testicular, and ovarian tumor cells, etc. Example biopsy-derived tumor organoids include, but are not limited to, mesothelioma, colorectal, appendiceal, lung, melanoma, and sarcoma organoids. In some embodiments, the cells include benign cells (also referred to as non-cancerous cells) obtained from a tissue biopsy. The cells may be differentiated at least in part to a particular cell or tissue type, such as brain, liver, intestine, pancreas, lymph node, smooth muscle, skeletal muscle, central nerve, peripheral nerve, skin, immune system, etc. Biopsy-derived cells (e.g., tumor and/or benign) may be used to form and/or prepare an organoid of the present invention, and the resulting organoid may be prepared and/or used in a method and/or device of the present invention within about 1, 2, 3, 4, 5, 6, 7, or 8 days after the biopsy.

In some embodiments, the cells of the organoid may be labeled with a detectable compound. A detectable compound may be any suitable compound that provides and/or generates a detectable signal that allows for differentiation and/or identification of a cell and/or cell population. A detectable signal may be provided and/or generated by one or more detectable compounds associated with a cell. In some embodiments, the detectable signal is a signal (e.g., an optical and/or electrical signal) that is generated by one or more detectable compounds (e.g., chemicals, proteins, etc.) associated with (e.g., applied to, attached to, bound to, compounded with, etc.) a cell. A detectable signal may be optically and/or chemically detectable, which may be perceived visually with the human eye and/or electronically read, detected, and/or obtained using methods known to those of skill in the art. In some embodiments, a detectable signal for a cell and/or cell population may be the absence of a signal (i.e., no detectable signal such as, e.g., no detectable fluorescence from the cell). In some embodiments, a detectable signal for a cell and/or cell population is a fluorescent compound (e.g., dye, protein, etc.). In some embodiments, the detectable compound may be used, for example, to track cellular or organoid growth and/or cellular or organoid movement.

The live tumor cells or cells obtained from a subject having a disease or other condition involving an accumulation of myeloid cells and the at least one type of myeloid cell may be present in an organoid in any suitable ratio. In some embodiments, the live tumor cells or cells obtained from a subject having a disease or other condition involving an accumulation of myeloid cells and the at least one type of myeloid cell may be present in the organoid in a ratio of about 1:1 or 5:1 to about 10:1, 20:1, 30:1, 40:1, 50:1, 60:1, 70:1, 80:1, 90:1, or 100:1 (tumor cells or cells obtained from a subject having a disease or other condition involving an accumulation of myeloid cells::myeloid cells). In some embodiments, the ratio of tumor cells or cells obtained from a subject having a disease or other condition involving an accumulation of myeloid cells to immune cells may be about 1:1, 2:1, 3:1, 4:1, 5:1, 6:1, 7:1, 8:1, 9:1, or 10:1. In some embodiments, the live tumor cells or cells obtained from a subject having a disease or other condition involving an accumulation of myeloid cells and the at least one type of myeloid cell are present in the organoid in a ratio of about 5:1, 10:1, 50:1, or 100:1.

In some embodiments, populations of cells obtained from a subject (e.g., from a tissue sample and/or biopsy from a subject) may be separated from one another to provide one or more separate populations of cells, and one or more of the separate populations of cells may be labeled and/or used to prepare an organoid as described herein. Methods of separating different populations of cells are known to those of ordinary skill in the art and any suitable method may be used, such as, but not limited to, fluorescence activated cell sorting (FACS) (Liao et al., J Vis Exp. 2016 Nov. 4; (117):54641; Bonner et al., Rev. Sci. Instrum. 43, 404-409 (1972); Vall et al., Fertil Steril. 2014 Aug; 102(2):566-580.e7). When two or more populations of cells are labeled, the two or more populations of cells may have a different detectable signal. In some embodiments, the tissue sample and/or biopsy may be genetically sequenced in part or in full in order to identify mutations, and any mutations identified may indicate and/or suggest one or more compound(s) of interest for therapeutic purposes for the subject (e.g., immune system modulating activity and/or anti-tumor activity).

The populations of cells (each of which may optionally be labeled) may be combined in any suitable manner to form the organoid. In some embodiments, the one or more populations of cells may be added to the same common media and/or hydrogel. In some embodiments, the one or more populations of cells may be used to form an organoid as described herein that is encapsulated by a hydrogel of the present invention. One or more different populations of cells in an organoid of the present invention may be present in substantially the same (e.g., within about ±20%) amount as the amount of cells in that population in a tissue and/or tumor in vivo.

In some embodiments, when cells have been obtained from a tissue sample from a subject, sorted and/or labeled, the different populations of cells are combined in substantially the amount as the amount present in the tissue sample. In some embodiments, cells used for an organoid of the present invention may not be sorted, which may aid in providing an organoid having native heterogeneity. For example, in some embodiments, cells are not sorted prior to forming an organoid and/or mixed organoid of the present invention, which may allow the organoid to mimic the native tissue heterogeneity by including, for example, any stromal cells, endothelial cells, immune cells, etc. present in the original biospecimen and/or sample.

In some embodiments, the myeloid cells and optionally, a second population of other cells, are mixed with warm (about 48° C.) molten agarose and/or gelatin to form a suspension of cells within the hydrogel (i.e., molten agarose and/or gelatin). The cell-agarose suspension may then be added (e.g., via pipette) into appropriately sized molds until solidified, then the solidified gels may be removed from the mold and placed in a complete growth medium at room temperature and maintained according to cell culture techniques that are well known in the art. Preferably, the hydrogel comprises about 0.1% to about 2.5% agarose and optionally, about 0.1% to about 2.5% gelatin.

In some embodiments, a hydrogel may comprise a plurality of organoids that may be the same or different than one another. For example a hydrogel may comprise 1 organoid to about 5,000 organoids, about 10 organoids to about 5000 organoids, about 100 organoids to about 5000 organoids, about 500 organoids to about 5000 organoids, about 1000 organoids to about 5000 organoids, about 1500 organoids to about 5000 organoids, about 2000 organoids to about 5000 organoids, about 2500 organoids to about 5000 organoids, about 3000 organoids to about 5000 organoids, about 3500 organoids to about 5000 organoids, about 4000 organoids to about 5000 organoids, about 4500 organoids to about 5000 organoids, or greater than 5,000 organoids. The volume of the hydrogel may be increased or decreased depending on the desired number of organoids in the hydrogel. As noted, the hydrogel may comprise one or more of each of the organoids described herein.

The disclosure also provides for methods of testing the efficacy of various therapeutic or new potentially therapeutic agents, as well as the effects of mechanical stress and microgravity on the disclosed organoids. For example, in some embodiments, a method of using a 3D in vitro model for testing mechanical stress on immune cells may comprise the steps of applying a compressive force to at least one organoid comprising a population of myeloid cells and determining a response to the compressive force by the population of myeloid cells as compared to a reference or control organoid that is not contacted by the compressive force or the same organoid but prior to applying the compressive force. Preferably, the organoid comprises the various cell populations and a hydrogel comprising agarose and/or gelatin where the organoid is embedded within the hydrogel.

In some embodiments, a method of using a 3D in vitro model for testing mechanical stress on immune cells may comprise the steps of applying a compressive force to at least one organoid comprising a population of myeloid cells and one or more other cell populations, and determining a response to the compressive force by the population of myeloid cells and/or the one or more other cell populations as compared to a reference or control organoid that is not contacted by the compressive force or the same organoid but prior to applying the compressive force.

In some embodiments, a method of using a 3D in vitro model for testing the efficacy of one or more agents of interest in treating a disease, or screening one or more agents of interest for immunological activity and/or modulating the immune system may comprise the steps of contacting at least one organoid comprising a population of myeloid cells with the one or more agents of interest, and determining a response to the one or more agents of interest by the population of myeloid cells as compared to a reference or control organoid that is not contacted by the one or more agents of interest or the same organoid but prior to contact with the one or more agents of interest.

In some embodiments, a method of using a 3D in vitro model for testing the efficacy of one or more agents of interest in treating a disease, or screening one or more agents of interest for immunological activity and/or modulating the immune system may comprise the steps of contacting at least one organoid comprising a population of myeloid cells and one or more other cell populations with the one or more agents of interest, and determining a response to the one or more agents of interest by the population of myeloid cells and/or and the one or more other cell populations as compared to a reference or control organoid that is not contacted by the one or more agents of interest or the same organoid but prior to contact with the one or more agents of interest.

In some embodiments, a method of using a 3D in vitro model for testing the effects of microgravity may comprise the steps of exposing at least one organoid comprising a population of myeloid cells and optionally, one or more other cell populations to microgravity and determining a response to the exposure to microgravity as compared to a reference or control organoid that is not exposed to microgravity. Exposure to microgravity includes various methods of space travel such as in a space station, a space shuttle, a space probe, or rocket or similar devices, or experience weightlessness, or zero g-force. In some embodiments, organoids exposed to microgravity also may be subjected concurrently or sequentially to compressive force and screened for compounds that treat diseases or screening one or more agents for immunological activity and/or modulating the immune system as described herein.

In some embodiments, the amount of compressive force applied to the hydrogel comprising the organoid is about 0.01 kPa to about 150 kPa, or about 1 kPa to about 100 kPa, or about 5 kPa to about 50 kPa. In some embodiments, the amount of compressive force applied to the hydrogel comprising the organoid is about 0.01 kPa to about 1 kPa, about 0.05 kPa to about 0.75 kPa, about 0.1 to about 0.5 kPa, or about 0.15 kPa. In some embodiments, a response from the population of myeloid cells in the organoid comprises one or more of an increase or decrease of the organoid size, an increase or decrease in the number of myeloid cells, an increase or decrease of a myeloid cell marker, an increase in activity of the myeloid cells and/or activation of the myeloid cell, and an increase or decrease of myeloid cell migration.

In some embodiments, a response from the population of macrophage cells in the organoid comprises one or more of an increase or decrease of the organoid size, an increase or decrease in the number of macrophage cells, an increase or decrease of a macrophage markers, an increase in activity of the macrophage cells and/or activation of the macrophage cell, an increase or decrease of macrophage cell migration.

In some embodiments, a response from the other cell population in the organoid comprises one or more of an increase or decrease of the organoid size, an increase or decrease in the number of other cells, an increase or decrease of a cellular marker, an increase in activity of the other cells and/or activation of the other cells, or an increase or decrease of the other cells migration.

In other embodiments, a positive response to the stimulus may include an increase in the size of a myeloid organoid and/or an increase in the number of myeloid cells in a myeloid organoid. In some embodiments, a positive response to the stimulus may include a decrease in the size of an organoid and/or a decrease in the number of certain cells present in the organoid, such as those that mimic a disease or condition, or for example, diseased cells or cancer cells. In some embodiments, a positive response to the stimulus may include an decrease in the number of myeloid cells and a decrease in the number of diseased cells. In some embodiments the positive (or negative) response to the stimulus is a comparison of the organoid prior to being exposed to the stimulus or to an organoid not subjected to the stimulus. In some embodiments, a positive response to the stimulus may comprise an increase in the amount of dead cells such as the myeloid cells, the diseased cell, or the cancer cells.

In some embodiments, the effect (i.e., response) to a compressive force, the efficacy of an agent of interest, and the effects of microgravity can be examined by measuring an increase or decrease in one or more biological markers selected from Piezo1, Trpv4, Nos2, Arg1, Ki67, Caspase 3 and GAPDH wherein the increase or decrease is measure using quantitative polymerase chain reaction (PCR), real-time PCR, serial analysis of gene expression (SAGE), microarrays, and/or Northern Blotting. Protein expression of the same may be determined using, for example, western blotting, 2-D electrophoresis, and immunoassays (Vandesompele et al. (2002), Genome Biol 3, research0034.1. PMID: 12184808; Guénin et al. (2009), J Exp Bot 60, 487-493. PMID: 19264760; Kitchen et al. (2010), Methods 50, 231-236. PMID: 20109551; Yuan et al. (2006), BMC Bioinformatics 7, 85. PMID: 16504059).

In some embodiments, the response to a compressive force, the efficacy of an agent of interest, and the effects of microgravity can be examined by measuring an increase or decrease in cell number and/or by viability of cells (live/dead), number of viable cells, number of viable cells versus dead cells. The status of the cells (i.e., live/dead tracking) may be monitored, for example, by Calcein-AM and Propidium Iodide staining (Buller et al., Blood. Volume 108, Issue 11, 16 Nov. 2006, Page 3879; Schmid et. al, (2001), Journal of Immunological Methods, 247, Issue 1-2, pp. 175-186; Kuonen et al. (2010), Cytometry, 77A: 1082-1090.).

In some embodiments, the response to a compressive force, the efficacy of an agent of interest, and the effects of microgravity can be examined by measuring metabolic activity using, for example, a (3-(4, 5-dimethylthiazolyl-2)-2,5-diphenyltetrazolium bromide (MTT) assay (Meerloo et al., Methods Mol Biol. 2011:731:237-45). This assay may be used, for example, to broadly measure the in vitro cytotoxic effects of an agent of interest on an organoid. Other cell viability assays that may be used to measure a response are described in Markossian S, Grossman A, Baskir H, et al., editors. Assay Guidance Manual [Internet]. Bethesda (MD): Eli Lilly & Company and the National Center for Advancing Translational Sciences; 2004, www.ncbi nlm.nih.gov/books/NBK144065/.

In some embodiments, the response to a compressive force, the efficacy of an agent of interest, and the effects of microgravity can be examined by immunohistological staining as described, for example in Beisker et al., (1987) Cytometry 8:235-239; Cowen et al. (1985) Histochemistry 82:205-208; Mosiman et al. (1997) Cytometry 30:151-156; and Romijn et al. (1999) J Histochem Cytochem 47:229-236.

In some embodiments, the response to a compressive force, the efficacy of an agent of interest, and the effects of microgravity can be examined by measuring the increase or decrease in size of the organoid, and/or an increase or decrease in the size or number of one or more populations of cells within the organoid. For example, the size or number of cells of an organoid comprising a population of myeloid cells may be measured, then the organoid may be exposed to a compressive force, an agent of interest, or the effects of microgravity, for a certain duration of time, and the organoid may be remeasured to determine an increase or decrease in the size of the organoid and/or an increase or decrease in the number of cells in organoid compared to prior to being exposed to a compressive force, an agent of interest, or the effects of microgravity.

In some embodiments, a response to a stimulus such as compressive force, an agent of interest, or exposure to microgravity is a decrease in the size of the organoid and/or a reduction in the number of live cells of the organoid (e.g. an increase in dead cells of one or both of the myeloid population and the other population of cell, such a tumor cells or diseased cells).

In some embodiments, the stimulus comprises the compressive force, and the measuring an immunological and/or physical response of the organoid comprises determining an amount of solid stress on the organoid based on a degree of deformation of the hydrogel surrounding the organoid, and at least one mechanical property of the hydrogel. For example, the mechanical property can be the elastic modulus, the tensile modulus, stiffness of the hydrogel, and the amount of agarose and/or gelatin in the hydrogel.

In some embodiments, the response to the compressive force, an agent of interest, or the effects of microgravity may be measured about 5 minutes to about 72 hours post application of the stimulus. In some embodiments, the response to the compressive force, an agent of interest, or the effects of microgravity may be measured about 5 minutes to about 72 hours, about 30 minutes to about 72 hours, about 1 hour to about 72 hours, about 5 hours to about 72 hours, about 10 hours to about 72 hours, or about 12 hours to about 72 hours post application of the stimulus. In some embodiments, the response to the compressive force, an agent of interest, or the effects of microgravity may be measured about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 36 hours, about 48 hours, or about 72 hours post application of the stimulus. In some embodiments, the response to the compressive force, an agent of interest, or the effects of microgravity may be measured about 1 day to about 10 days, about 1 day to about 7 days, or about 3 days to about 5 days hours post application of the stimulus. In some embodiments, the response to the compressive force, an agent of interest, or the effects of microgravity may be measured about 1 day to about 4 days, about 5 day to about 7 days, or about 8 days to about 14 days post application of the stimulus. In some embodiments, the response to the compressive force, an agent of interest, or the effects of microgravity may be measured within about 1 week, about 2 weeks, about 3 weeks, about 4 weeks, about 5 weeks, or about 6 weeks (e.g., within about 2, 3, 4, 5, 6, 7, 8, 9, 10, 11, 12, 13, 14, 21, 28, 35, or 42 days) post application of the stimulus.

Agents of interest may include, for example, an enzyme, a protein, a polynucleotide, an antibody or the like, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds (anti-viral, anti-bacterial, immunotherapeutic, chemotherapeutic, etc.). Other agents of interest are described, for example, in U.S. Pat. No. 11,116,848 to Bilgicer et al., and U.S. Patent Publication No. 2021/0087250 to Werle et al.

As noted, some embodiments, the organoids of the invention can be used to test libraries of chemicals, antibodies, natural product (plant extracts), etc. for suitability for use as drugs, cosmetics and/or preventative medicines. For instance, in some embodiments, a cell biopsy from a patient of interest, such as cells from a subject having an active Mycobacterium tuberculosis infection or other disease or conditions having an accumulation of myeloid cells, and in particular, an accumulation of macrophages, can be cultured and formed into an organoid and then treated with a chemical compound or a chemical library. It is then possible to determine which compounds effectively modify, kill and/or treat the subject's cells. This allows specific patient responsiveness to a particular drug to be tested thus allowing treatment to be tailored to a specific patient. Thus, this allows a personalized medicine approach. The added advantage of using the organoids for identifying drugs in this way is that it is also possible to screen normal organoids (organoids derived from healthy tissue) to check which drugs and compounds have minimal effect on healthy tissue. This allows screening for drugs with minimal off-target activity or unwanted side-effects. Thus, in some embodiments, a method of screening for a therapeutic or prophylactic drug comprises contacting an organoid embedded in a hydrogel as described herein to one or a library of candidate molecules, evaluating the organoid for any response, for example any change in the cell, such as a reduction in or loss of proliferation, a morphological change and/or cell death, identifying the candidate molecule that causes the response as a potential drug, and optionally providing said candidate molecule, e.g. as a drug. In other embodiments, a method of screening for a therapeutic or prophylactic drug comprises contacting an organoid comprising one or more population of cells derived from a biological sample of a subject that is embedded in a hydrogel as described herein to one or a library of candidate molecules, evaluating the organoid for any response, for example any change in the cell, such as a reduction in or loss of proliferation, a morphological change and/or cell death, identifying the candidate molecule that causes the response as a potential drug or cosmetic, and optionally providing said candidate molecule, e.g. as a drug or cosmetic. Other methods of screening for therapeutic agents are described, for example, in U.S. Pat. No. 12,331,314 to Ortega et al.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Novel 3-D Macrophage Spheroid Model Reveals Reciprocal Regulation of Immune-Mechanical Stress and Mechano-Immunological Response

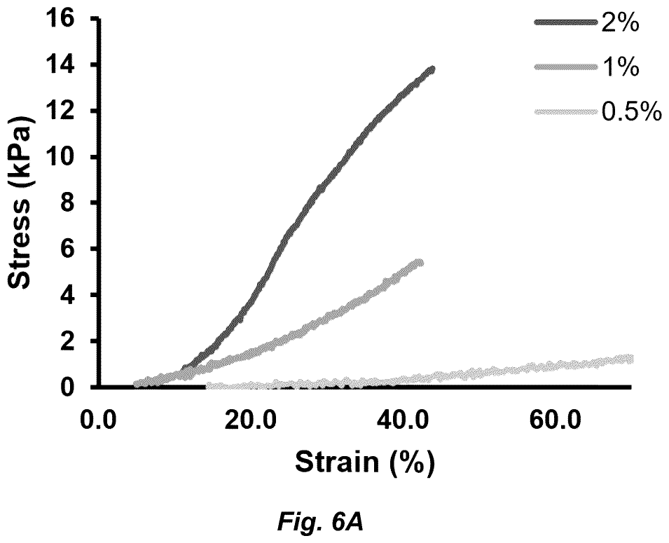

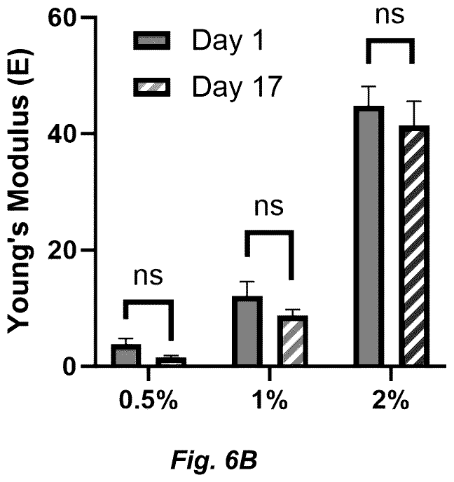

Agarose-embedded macrophages form spheroids with long-term viability. To determine whether macrophages alone can generate solid stress, we utilized an agarose hydrogel to serve as a confining gel. As a plant-derived material, agarose is biologically inert to mammalian cells, and is also physically and chemically stable, with suitable biocompatibility. The mechanical properties of agarose are also easily tunable, as Young's modulus increases exponentially with molar concentration. We measured the stiffness of 0.5%, 1%, and 2% low melting point cell culture grade agarose using an unconfined compression testing device to obtain stress-strain curves (FIG. 6a). From these, we calculated the average Young's modulus at each concentration. After one day of equilibration, 0.5%, 1%, and 2% agarose had average Young's moduli of 3.8 kPa, 12.1 kPa, and 44.8 kPa, respectively. These approximately correspond to the stiffness ranges measured in the brain, healthy heart, and fibrotic scar tissue, respectively. We tested gels that had been equilibrated in media under standard cell culture conditions for either one day or 17 days, finding no obvious change in material properties over time (FIG. 6b).

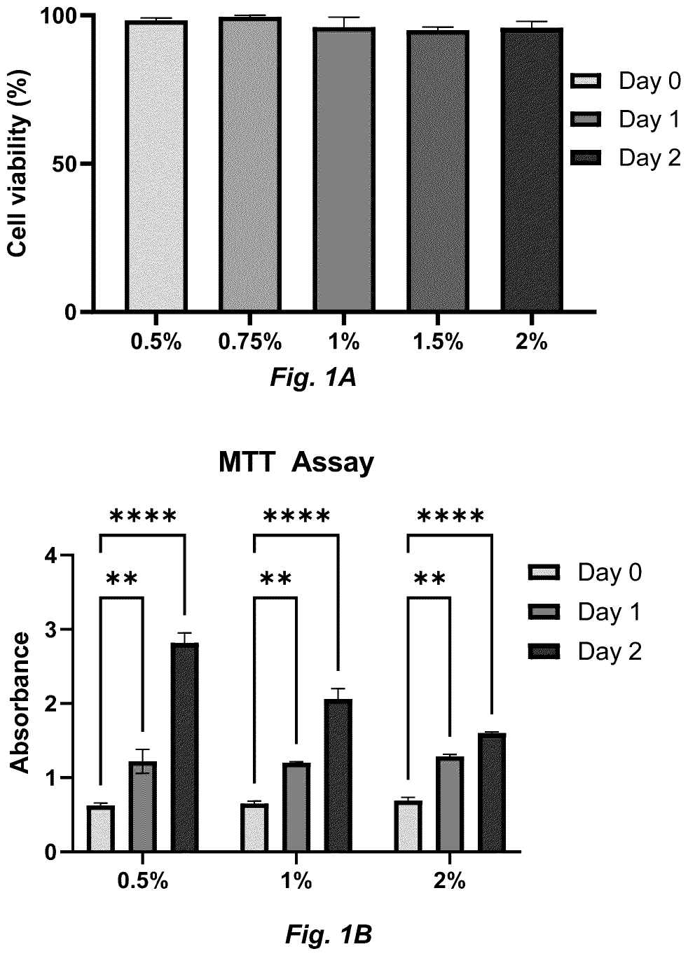

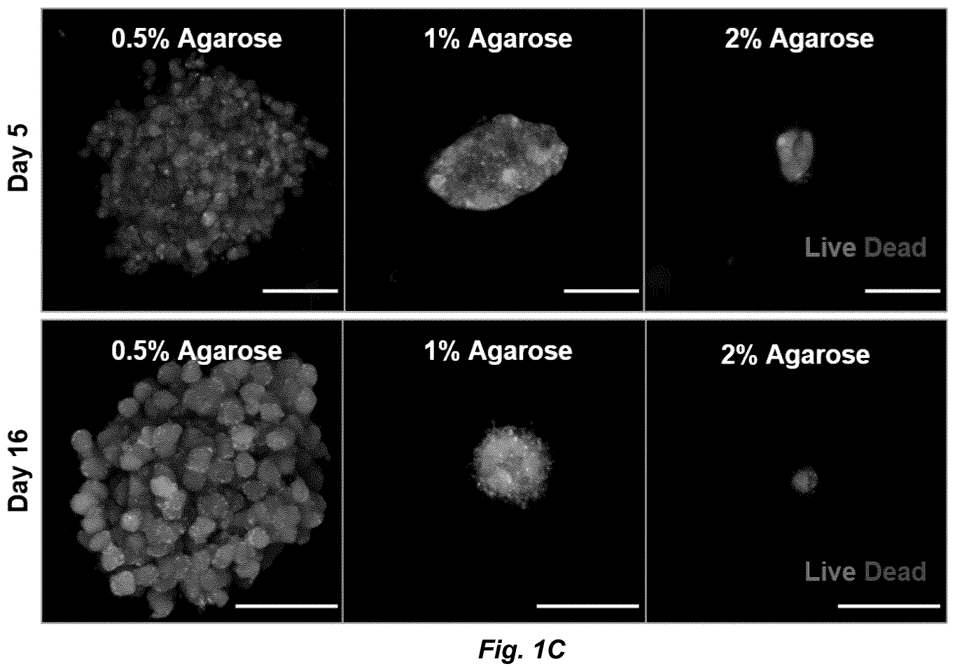

Having determined that agarose is mechanically suitable for disease modeling, we next verified that it was biocompatible with our RAW264.7 murine macrophage cell line. Because the agarose embedding process involved mixing live cells with warm molten agarose, we tested whether the embedding process would result in cell death. We found that macrophages embedded in agarose between the concentrations of 0.5% and 2% had nearly 100% viability at each concentration with no significant difference between conditions, as measured one day after embedding via calcein-AM and PI staining (FIG. 1a). The metabolic activity of agarose-embedded cells measured in an MTT assay increased significantly over time in all three agarose concentrations (FIG. 1b). Metabolic activity was uniform between 0.5%, 1%, and 2% agarose initially after embedding and one day after embedding. By day 2, however, cell proliferation increased total metabolic activity per gel, with softer gels having a larger increase in activity compared to stiffer gels.

Over time, the agarose-embedded macrophages proliferated to form “spheroids,” or clumps of multiple cells that expand in place (FIG. 1c). We intentionally chose a material that the cells could not manipulate, as we sought to assess the ability of macrophages to generate growth-induced solid stress through expansion. Any cell proliferation must therefore cause solid stress, as the cells must displace the elastic material to make room for the growing 3D structure. Agarose-embedded macrophage spheroids displayed excellent long-term viability, with healthy spheroids observed 16 days after seeding (FIG. 1C). For longer-term culture, cells were seeded at a lower density, and as a result grew more slowly, resulting in smaller spheroids observed on day 16 compared to larger spheroids observed at earlier timepoints when seeded at a higher density. Live spheroid constructs can likely be maintained for much longer periods than reported here, as we observed the time in culture to be limited only by the eventual overgrowth of the gel by cells.

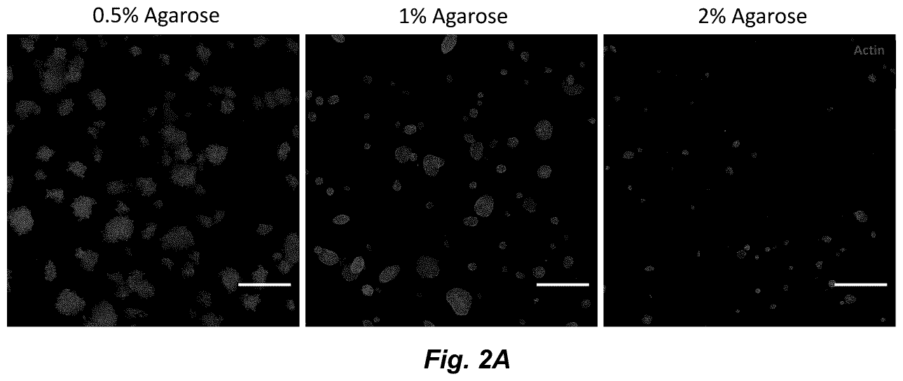

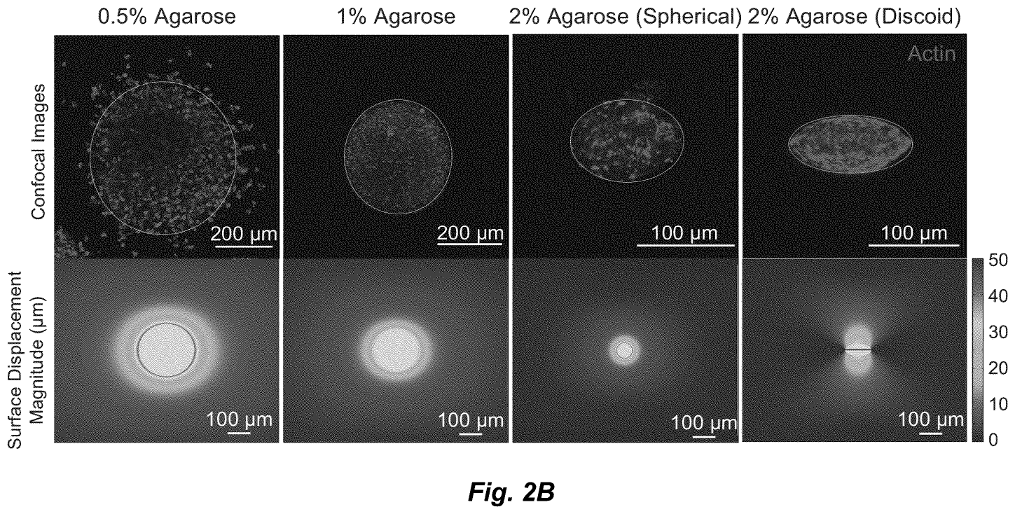

Macrophages generate solid stress in confining agarose gels. The 3D morphology of the spheroids was captured via confocal imaging, which reveals a variety of shapes, not only spheroidal aggregates. In the softer 0.5% gel, individual cell profiles were visible as they protruded into the surrounding gel, while stiffer gels tended to result in smoother spheroid edges (FIG. 1c). Due to the computational complexity of modeling an exact 3D morphology, we completed our solid stress modeling using 2D maximum intensity projections of spheroids stained for actin (FIG. 2a).

Given an estimate of the spheroid geometry and the mechanical properties of the gel, we generated a simulation of the displacement field in the gel surrounding the spheroids using COMSOL (FIG. 2b). Simulations of spheroid displacement during growth are shown based on the corresponding fluorescent images. In FIG. 2b from left to right, the first three simulations for a spheroid embedded in 0.5%, 1%, and 2% agarose gels assume no microcrack developments in the gels and an initial uniformly spherical geometry with a 5 μm radius, while the simulation for the spheroid in the 2% agarose gel with a discoid shape assumes formation and propagation of a microcrack in the gel during initial spheroid growth, as observed in similar agarose-embedding studies. The greatest solid stress-induced deformations are observed in 0.5% gels (with maximum gel displacements of ˜250 μm), compared to stiffer 2% gels with spherical (40 μm) and discoid (42 μm) shapes. However, solid stress propagates further into the surrounding matrix with increasing gel concentrations. These data demonstrate that increasing the rigidity of the surrounding agarose matrix by elevating gel concentration significantly restricts spheroid expansion and modulates growth morphology. The decreased gel displacement and altered shape in stiffer gels indicate that the increased mechanical resistance of the matrix impedes outward growth. In contrast, the more mechanically permissive and compliant 0.5% agarose gel allows for greater spheroid expansion which maintains a rounded shape.

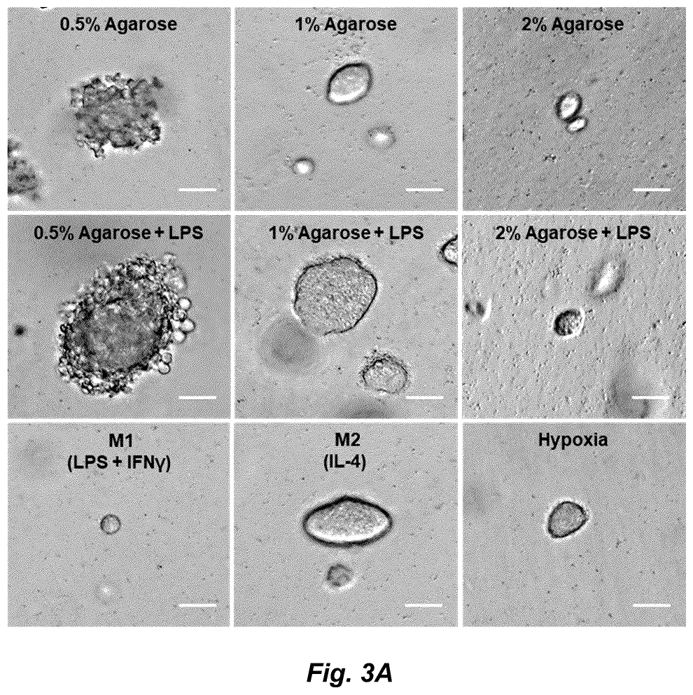

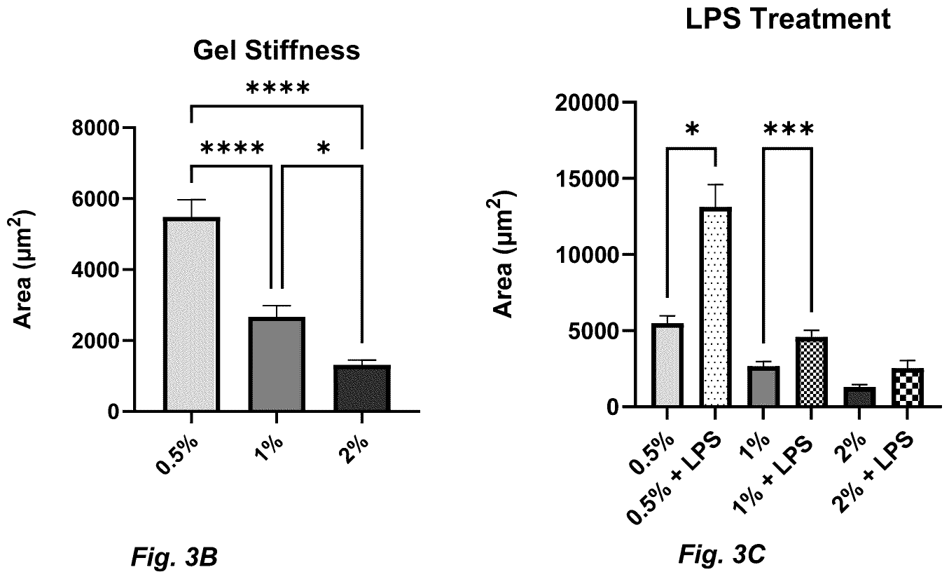

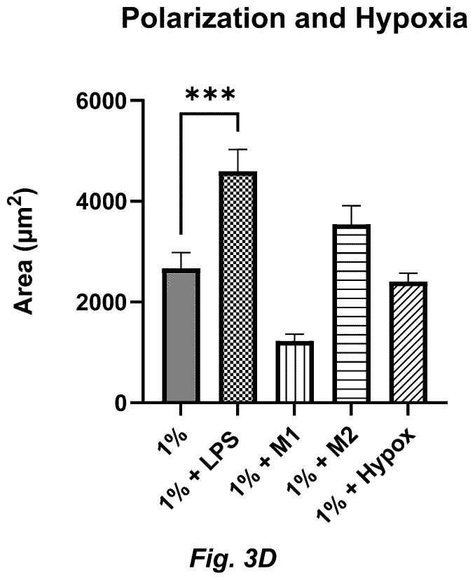

Spheroid size decreases with increasing agarose concentration and increases with pro-inflammatory stimulation. After establishing that agarose can both support healthy cell culture and enable the generation of solid stress, we next explored the effects of various biologically relevant stimuli on spheroid size (and therefore stress generation). In addition to the clearly visible effects of agarose concentration (FIG. 3a), chemical stimuli added to the culture media also influenced spheroid size. The addition of 200 ng/ml lipopolysaccharide (LPS) serves to simulate a bacterial infection, as it is derived from a common bacterial fragment and is known to elicit potent macrophage responses. To induce the canonical pro-inflammatory M1-like polarization of macrophages (denoted “M1” in the figure), the cultures were treated with 200 ng/ml LPS plus 20 ng/ml interferon-γ (IFNγ). For canonical anti-inflammatory M2-like polarization (denoted “M2”), cells were treated with 20 ng/ml interleukin-4 (IL-4). Aggregates larger than singlet or doublet cells created an easily visible outline in phase-contrast images and their sizes were compared based on the spheroid area. We found that metrics such as solidity and eccentricity (measures of smoothness and elongation, respectively) had no meaningful differences between the tested conditions (data not shown).

Spheroid size was significantly increased in 0.5% agarose compared to 1% agarose and was significantly reduced in 2% agarose compared to 0.5% (FIG. 3b). Thus, spheroid growth appeared to be inhibited with increasing matrix stiffness. Adding LPS increased spheroid size, particularly in 0.5% and 1% conditions (FIG. 3c). As agarose stiffness increased, the effect of LPS diminished, and the change in size observed in 2% agarose with the addition of LPS was not statistically significant. M1-like polarization caused a non-significant trend towards decreased spheroid size, consistent with the hypothesis that M1-like macrophages shift their metabolic state to favor effector immune function rather than proliferation (FIG. 3d). M2-like polarization caused a non-significant, slight increase in spheroid size. Hypoxia is a phenomenon observed in cancerous tumors (as well as granulomas and other tissue masses) and is predicted to contribute to the pathological phenotype of tumor-resident macrophages. However, the direct effect of hypoxia on macrophage mechanical behavior is unknown. Our data show that M1-like and M2-like polarization may have caused a slightly reduced or slightly increased spheroid size, respectively. Hypoxic macrophages may have an intermediate polarization state in which these two effects cancel out.

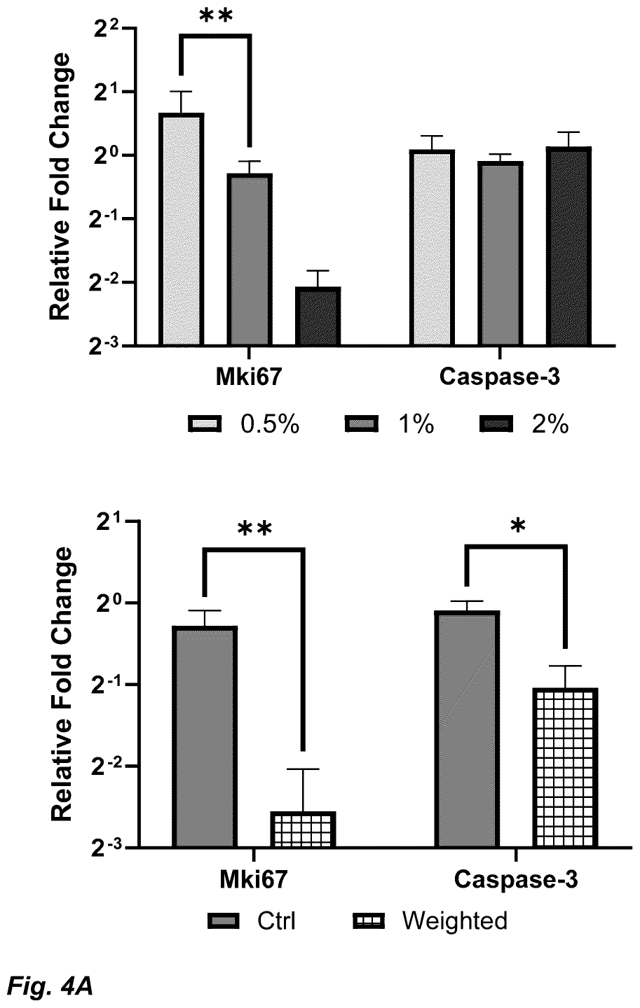

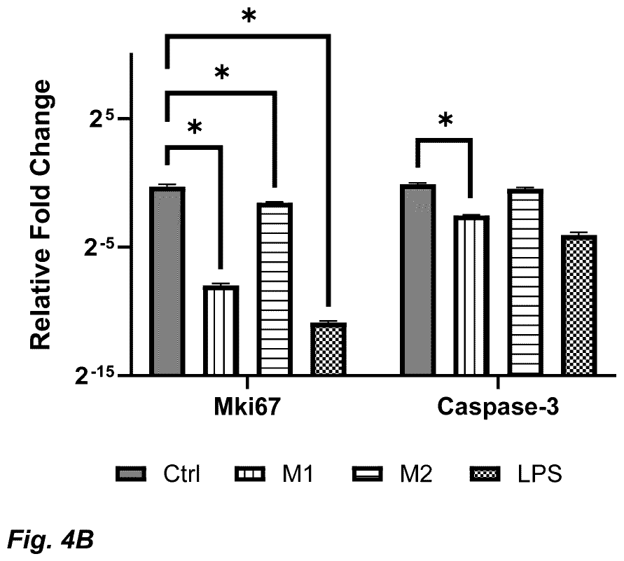

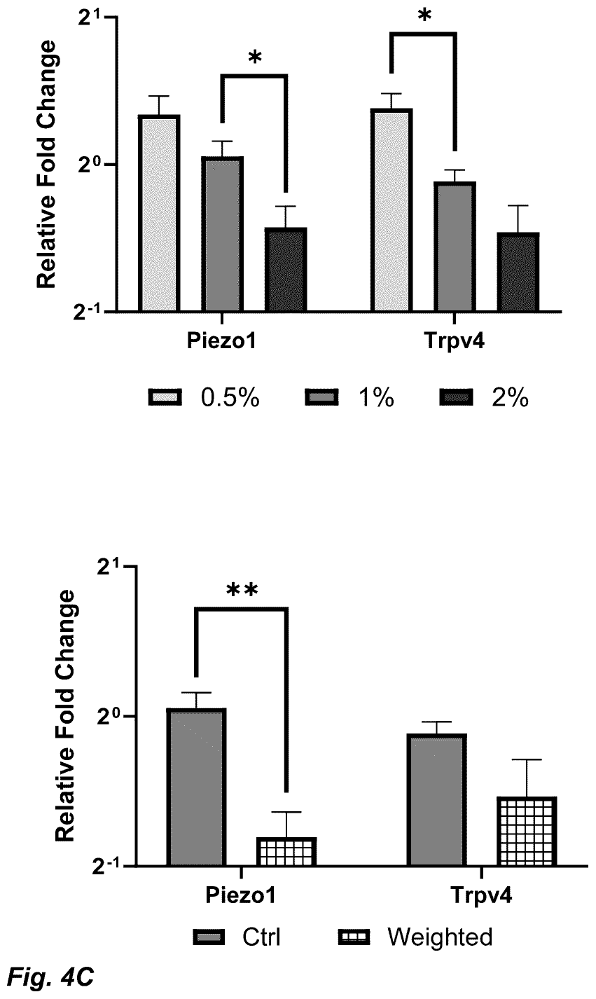

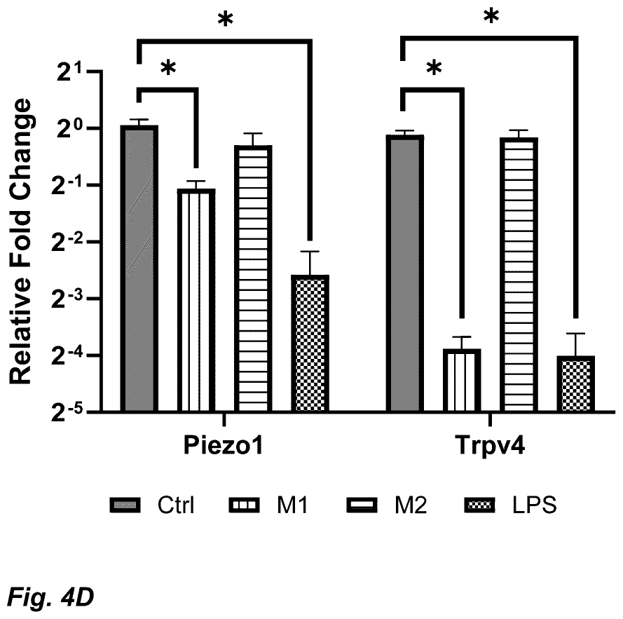

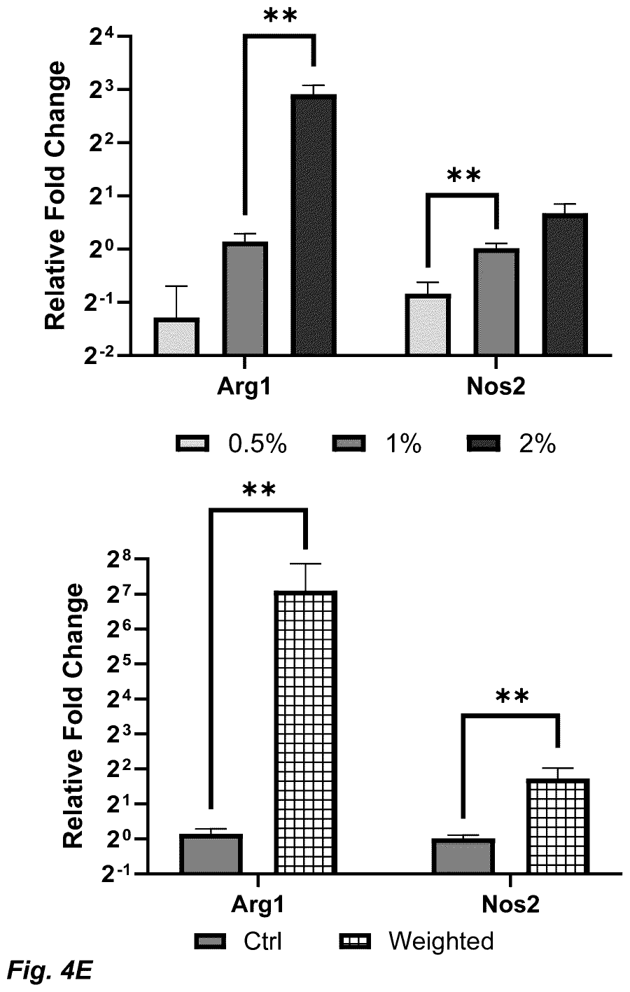

Macrophages in 3d gels display altered mechanical and inflammatory transcriptional responses to varied mechano-chemical stimuli. We next compared the transcriptional activity of macrophages under various mechanical and/or chemical conditions using qPCR. Mki67 (encoding Ki67) decreased significantly between 0.5% and 1% agarose, with a non-significant decrease from 1% to 2% (FIG. 4a-b). This aligns well with the spheroid size data (FIG. 3b), implicating reduced proliferation behind the observed reduced spheroid size in stiffer gels. There was no significant difference in Casp3 (encoding Caspase-3) expression with agarose concentration. This, together with the near-complete viaility observed via calcein-AM and PI staining, indicating that apoptosis is likely not responsible for the reduced size with increased stiffness.

We also tested the effect of externally applied compression, to determine whether compressive stress could impact the macrophages, potentially completing a macrophage-compression feedback loop. We applied 0.14 kPa of uniaxial compression, approximately the magnitude measured in murine glioblastoma models (Nia et al., Nat Biomed Eng 1:0004, doi.org/10.1038/s41551-016-0004; Nia et al., Nat Protoc 13:1091-1105., doi.org/10.1038/nprot.2018.020; Community SNP and M (2020) In vivo compression and imaging in the brain to measure the effects of solid stress. In: Springer Nature Protocols and Methods Community) This resulted in a significant decrease in Ki67 expression, and, surprisingly, a concurrent decrease in Casp3 (FIG. 4a-b). This indicates that a reciprocal regulation may exist between macrophages and the solid stress they generated, though this warrants further investigation. M1-like polarization, M2-like polarization, and LPS stimulation all significantly decreased Ki67 expression. This is interesting, given that the addition of LPS was observed to cause an increase in spheroid size. This suggests that either Ki67 gene expression is not an accurate reflection of cell proliferation, or that spheroid size may change independent of proliferation, such as through changes in individual cell sizes.

Piezo1 and Trpv4 (encoding Piezo1 and TRPV4, respectively) were also altered based on gel stiffness and chemical cues. Trpv4 expression decreased between 0.5% and 1% agarose, and decreased with both M1-like and LPS treatments (FIG. 4c-d). Piezo1 expression also significantly decreased with both M1-like and LPS treatment, though to a lesser degree. Piezo1 also decreased between 1% and 2% agarose, with a trending decrease between 0.5% and 1%. Reduced mechanosensor expression may indicate that a decreased sensitivity to confining solid stress may play a role in the increase in observed increase spheroid size between untreated and LPS-treated gels (FIG. 3c). Interestingly, applied compression caused a significant decrease in both Piezo1 and Trpv4 (FIG. 4bc-d).

Finally, we quantified canonical markers of pro-inflammatory M1-like macrophage activation (Nos2, encoding inducible nitric oxide synthase [iNOS]) and anti-inflammatory M2-like polarization (Arg1, encoding arginase 1) (FIG. 4e-f). We observed a trending increase in Nos2 and Arg1 with increased stiffness, with statistical significance between 1% and 2% for Arg1 and between 0.5% and 1% for Nos2. Both Arg1 and Nos2 increased significantly with externally applied compression. As expected, Nos2 increased with M1-like and LPS treatment and decreased with M2-like treatment. However, Argl unexpectedly increased with all three stimulation/polarization treatments, potentially indicating an altered phenotypic response to these stimuli in 3-D compared to previously observed 2-D culture results.

This 3-D model of macrophage spheroid formation and the accompanying computational modeling of stresses and strains and downstream cellular and molecular biology readouts combine to create a novel platform for investigating the immunomechanics and mechano-immunology of macrophages in varying biochemical and mechanical microenvironments. We chose to use low melting point agarose to leverage its mechanical tunability, chemical stability, and biological inertness. While this model lacks the adhesive qualities of other tissue-mimetic hydrogels, it enables us to isolate specific mechanical phenomena of interest and exclude confounding factors such as cell migration, changes in gel stiffness and structure due to cell activity, etc. As the spheroids grow into large multicellular structures, each cell still experiences cell-cell contact. The inner cells experience contact on all sides, as cells in the body do. The cells on the spheroid surface experience cell-cell contact on one side, with inert agarose on the other. This model is thus suited to capture basic mechanobiological phenomena.

To the best of our knowledge, this is the first demonstration of successful spheroid formation, long-term viability, and solid stress generation by macrophages alone. The agarose gel constructs are simple to generate, inexpensive, and reproducible, making them attractive for high-throughput applications, such as drug screening. The model is also highly amenable to more complex co-culture or organoid experiments, as any number of cell types and treatments can be incorporated. It could therefore be used to model solid stress in other disease settings, such as within tuberculosis granulomas where macrophages dominate, or tissue regeneration processes that involve mechanosensitive crosstalk between stromal cells and macrophages. Additionally, the agarose gels can be processed in the same way as tissues for histological analysis. We are currently optimizing methods for sectioning and staining for both cryopreservation and paraffin-embedding. This platform is also compatible with standard RNA extraction methods.

While many aspects of macrophage responses to various forms of mechanical stress have been studied, relatively little has been shown about the reciprocal regulation of macrophages and solid stress. Our simulation results suggest that the mechanical microenvironment can override intrinsic growth programs to control spheroid expansion. Softer gels allow for a greater displacement of the gel around the spheroid compared to higher-stiffness gels. However, in stiffer gels, the gel deformation relative to the size of the spheroid is higher. This work also reveals an interesting 3-D growth pattern, as macrophages often adopt a flattened discoid shape, rather than a spheroidal shape. This could indicate either a physical process, such as the formation of a planar crack in the gel, or a biological process, such as differential proliferation or tip/leader cell migratory behavior in different regions of the spheroid. Further work to characterize the agarose gel surrounding a spheroid and potential crack propagation is underway. A limitation of the computational model is that it provides an estimate of the displacement of the agarose adjacent to the spheroid, but not within the spheroid itself. Future work to characterize the mechanical properties of macrophage spheroids would inform computational modeling of stress distribution within the spheroids.

The most potent biochemical cue that increases macrophage spheroid size and stress generation at all tested agarose concentrations is treatment with LPS, an immunostimulatory bacterial fragment used to mimic the effect of bacterial infection. Interestingly, the addition of IFNγ along with LPS, a standard M1-like polarizing regime, reversed the effect of LPS entirely. IFNγ and LPS are generally thought to synergize to induce an M1-like phenotype, but this 3-D stress-generation model appears to have revealed a context in which the two oppose one another. This corroborates findings from a recent publication that found that IFNγ can counteract a subset of the effects of LPS stimulation in periphery human monocytes, underscoring the importance of the interaction of different environmental factors in macrophage function. Further mechanistic studies utilizing this model will help to elucidate the nuances of LPS-stimulated versus canonically M1-like polarized macrophages.

Mammalian cells in culture respond to hypoxic conditions within hours of exposure, with some pathways reaching maximum response in the 24-72 hour time range. Interestingly, hypoxia did not significantly alter spheroid size (FIG. 3d). The literature is divided on whether hypoxia will promote a more pro-inflammatory or anti-inflammatory macrophage phenotype, with some observing an increase in M1-associated pathways, and another observing an increase in M2-associated pathways. However, macrophage phenotype is now understood not to lie on a simple M1-M2 spectrum, but can take the form of intermediate states, with pathological macrophages often displaying aspects of both ends of the canonical spectrum.

Two prominent mechanosensory ion channels—Piezo1 and TRPV4—have been investigated previously in macrophage response to mechanical cues. When the cell membrane stretches, these channels are activated, causing calcium influx and resulting in the movement of transcription factors into the nucleus and activation of pathways related to macrophage polarization, proliferation, and function. We saw that macrophage TRPV4 expression decreased with increasing 3D stiffness, the opposite of what has been observed for mammary epithelial cells and chondrocytes in 2D settings. We also observed an increase in an M2-like marker with stiffness. Piezo1 expression by macrophages has been shown to increase on stiffer surfaces. However, we observed decreased Piezo1 expression with increased stiffness. Cells in a confining gel such as agarose that does not support cell adhesion may not experience the membrane tension can activate stretch-activated channels such as Piezo1. Applying uniaxial compression to the gel resulted in similar transcriptional responses as increasing stiffness for all markers except Caspase-3. The macrophages in the spheroids may therefore be sensing reciprocal mechanical forces rather than mechanical properties of their surroundings in the case of varying stiffness.

While macrophage phenotype can be modulated by integrin interactions with the extracellular matrix in 2-D and 3-D models, there are also important integrin-independent mechanosensing mechanisms. Recent work has shown that the mechanisms by which macrophages both sense and generate growth-induced mechanical forces such as solid stress are distinct from other traditionally adhesion-dependent cells. As opposed to adhesive cells such as fibroblasts, macrophages do not only rely on integrin adhesions to respond to extracellular matrix stiffness but rather respond via a distinct pathway involving cytoskeletal dynamics. Rather than “pulling” on the surrounding matrix, they push outwards through cellular protrusions, and the resulting compressive stress transduces information about the environment stiffness, leading to downstream responses. Macrophages also use an adhesion-independent “amoeboid” migration mode, relying on the expansion of the actin network in their leading edge to migrate, rather than producing traction forces via integrin adhesions. This results in forces that act to expand their substrate in the direction of motion, pushing the material both in front and behind, resulting in a net movement forward. In both cases, macrophages demonstrate their ability to both sense and respond to solid stress on a subcellular scale. Future work with high-resolution imaging of the macrophage cytoskeleton in these 3D constructs may elucidate this effect on the spheroid scale.

Macrophages have increasingly been a subject of interest as targets for disease treatments. A meta-analysis reports more than 25 clinical trials targeting tumor-associated macrophages in a range of different cancer types. In atherosclerosis, reducing macrophage mass within plaques is a promising strategy, as is increasing the numbers of anti-inflammatory macrophages and decreasing pro-inflammatory macrophages in damaged heart tissue after myocardial infarction. Depleting macrophages in models of skin wounding reduces hypertrophic scar formation. As previously shown, targeting either macrophages or solid stress in glioblastoma improves outcomes. Thus, understanding factors that contribute to macrophage expansion or reduction via a novel 3-D system could inform future treatment strategies.

Overall, this work demonstrates a novel platform to study previously unexplored aspects of macrophage mechanics. Because many diseases involve both altered macrophage content and altered mechanics, this model may elucidate new and targetable pathological interactions between macrophages and solid stress. Understanding how macrophages generate stress, and how they respond to external cues under chronic solid stress, will inform strategies to target or reprogram macrophages in the plethora of diseases that involve macrophage accumulation. This platform also has promise for screening macrophage-targeted drugs and is highly tunable to apply to a range of diseases.

### Example 2

### Generation and Analysis of Glioblastoma-immune Organoids

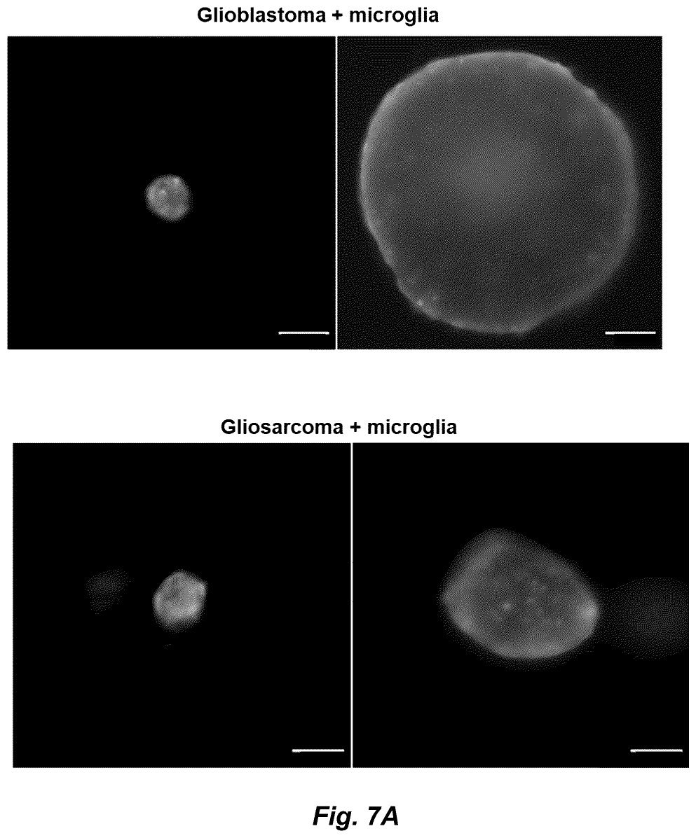

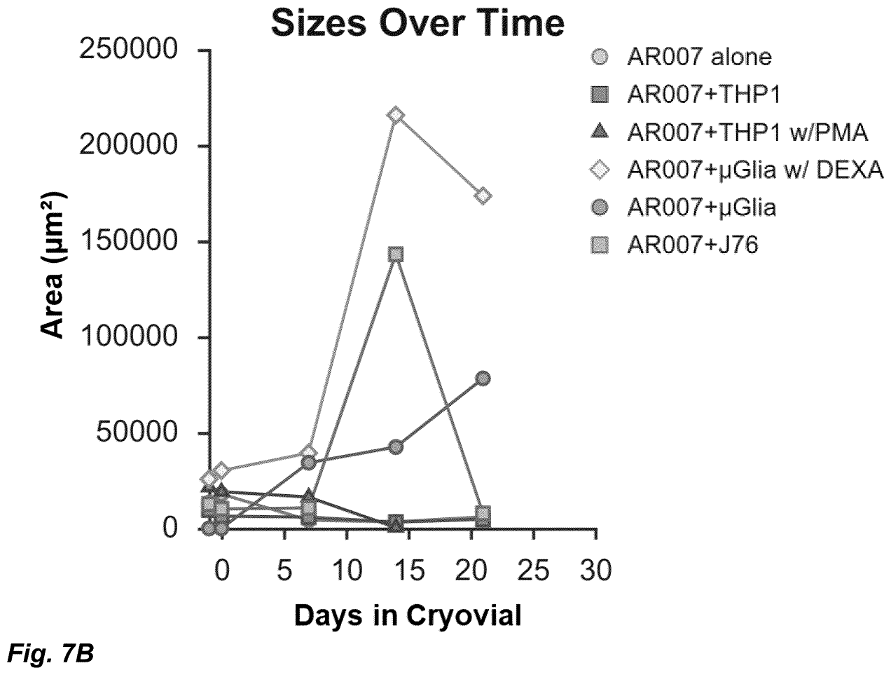

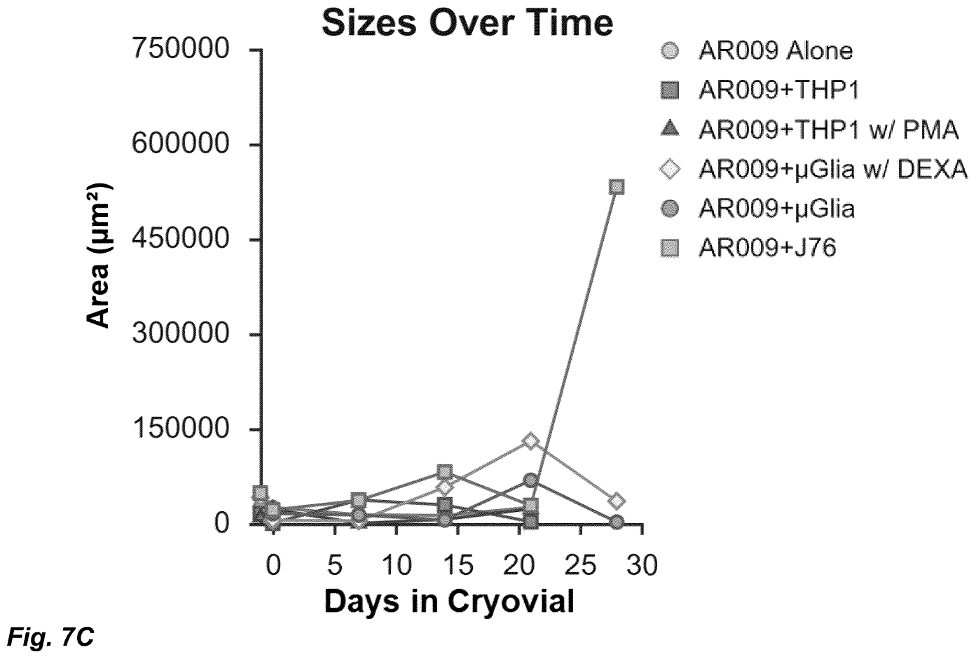

We have successfully developed organoid models using five patient-derived glioma cell lines in combinations with four different immune cells types. These organoids are formed using the hanging drop method (see, for example, U.S. Patent Publication No. 2014/0221225 to Danen et al; Hopfl et al., Methods Mol Biol 254:79-98 (2004)), embedded in an agarose gel, and cultured for several weeks in a sealed cryovial system compatible with the Space Tango Cubelab. Viability staining of representative organoids over time is shown in FIG. 7a. Quantification of organoid size over time shows differential growth rates of different glioma cell lines (AR007—glioblastoma, AR009-gliosarcoma) and immune combinations (THP1—monocyte, THP1+PMA—macrophage, uGlia—microglia, J76—T-cells) (FIG. 7b-c). We have collected time-course viability and size data for nearly 100 different organoid conditions. This data allows us to pinpoint ideal experimental conditions for short-term and long-term organoid experiments in space.

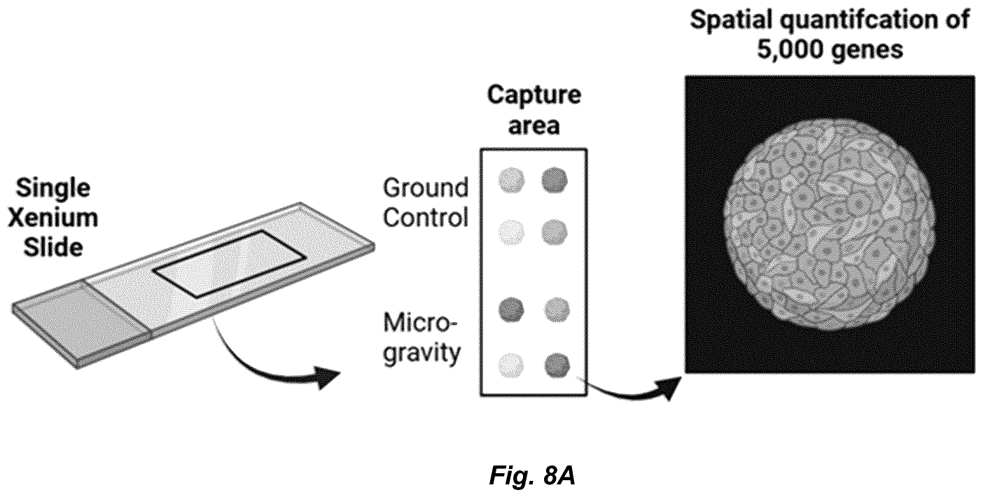

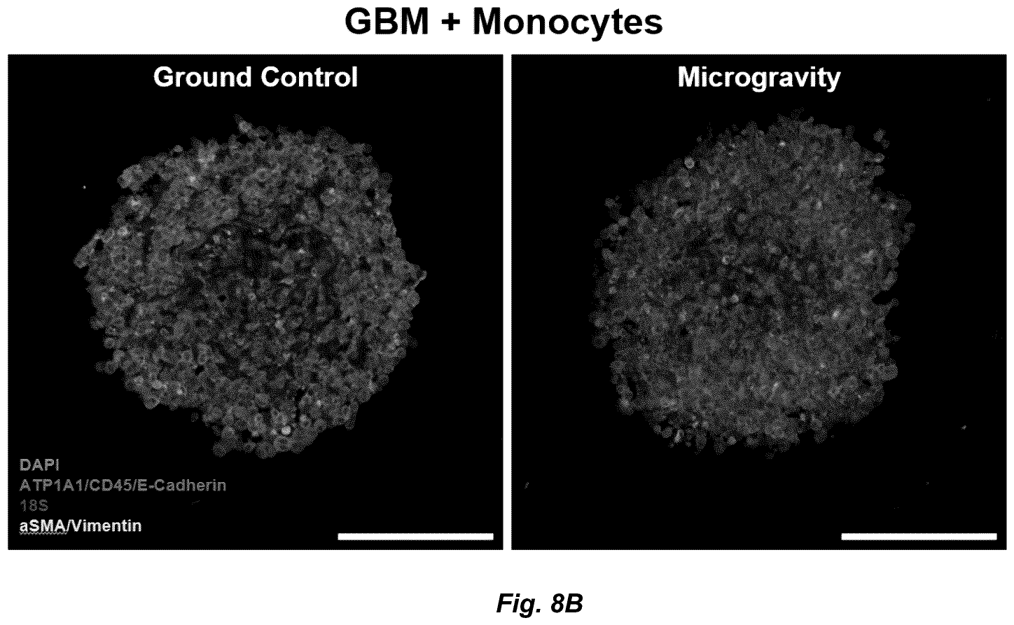

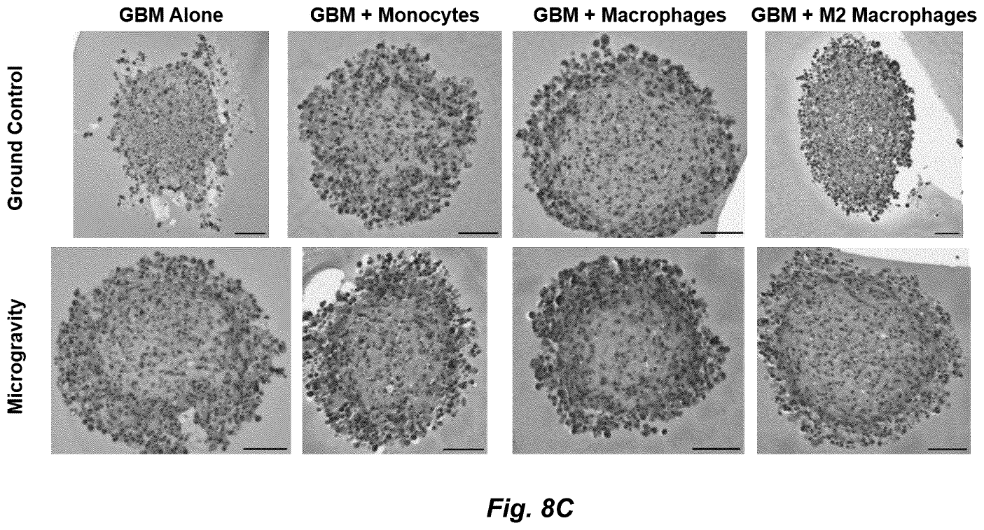

We have also successfully optimized protocols for organoid sectioning and downstream immunofluorescent, histological, and spatial transcriptomics analysis. We found that individual organoids embedded in a mixture of 2% agarose and 2% gelatin can be FFPE processed, reliably preserving morphology and preventing organoid loss. We used these methods to analyze organoids that were embedded in our agarose system and cultured on the International Space Station for 40 days, along with synchronous ground controls. We sectioned organoids with four distinct cellular compositions from our space and ground control experiments. These were processed using the 10× Genomics Xenium platform (FIG. 8a). This successfully produced immunofluorescent staining images (FIG. 8b). We also did Hematoxylin and Eosin staining for histological analysis on serial sections (FIG. 8c). Xenium transcriptomics analysis will be conducted with a 5,000-gene panel run on 8 distinct organoids on a single slide.

### Example 3

### Material and Methods

Cell culture, gel formation, and macrophage activation/polarization. RAW264.7 murine macrophages were purchased from ATCC (TIB-71). They were grown in a complete culture medium consisting of DMEM (Corning, 10-013-CV) supplemented with 10% Fetal Bovine Serum (Gibco, 26140079) and 1% penicillin-streptomycin (Corning, 30-002-CI). They were maintained in a humidified incubator at 37° C. with 5% CO₂. Cells were grown as adherent monolayers and passaged using a cell scraper for mechanical detachment.

To create agarose-embedded 3-D cultures, single-cell suspensions were mixed with low-gelling temperature agarose (Sigma-Aldrich, A0701-25G). First, a 4% agarose solution was made in a complete culture medium and heated in a microwave until dissolved. The liquid agarose was maintained at 48° C. until use. Suspensions of RAW264.7 cells in medium were mixed with a proportional amount of the 4% agarose to create gels with a final concentration of 0.5%, 1%, or 2% agarose and 1,000 cells/ml to 10,000,000 cells/ml, depending on the experiment. The cell-agarose solution was pipetted into 2 mm-deep cylindrical molds and left at room temperature to set for 10 minutes. The gels were then removed from the molds, submerged in complete culture medium with or without supplementation listed below, and maintained under standard culture conditions. The cylindrical molds were made using custom 3D-printed polylactic acid (PLA) rings adhered to a glass coverslip. These were sterilized with 70% ethanol and 30 minutes of UV light exposure in a biosafety cabinet prior to use.

The gels in each condition were treated immediately after the gel solidified so that the embedded cells experienced the various treatments for the entire time they were embedded in agarose. To simulate bacterial activation of macrophages, the medium was supplemented with 200 ng/ml lipopolysaccharide (LPS, Santa Cruz Biotechnology, sc-3535). For M1-polarization-treated gels, the medium was supplemented with 20 ng/ml IFN-γ (BioLegend, 575302) and 200 ng/ml LPS. For M2-polarization-treated gels, the medium was supplemented with 20 ng/ml IL-4 (Pepro-Tech, 214-14). The volume of the gel was included in the total solution volume to achieve accurate final concentrations. Gels cultured under hypoxic conditions were placed in a Tri-Gas hypoxia incubator with 5% CO₂ and 1% O₂. Compressed gels were placed on a 0.4 μm pore size transwell cell culture insert (CellQART, 9310402) and a 3-D printed PLA weight was placed on top to apply 0.14 kPa of compression to the gel to simulate the solid stress measured in murine glioblastoma models. The pores in the transwell membrane enable media exchange with media in the lower well, without allowing the transmigration of cells. Because compression results in anisotropic spheroid growth, and because our size analysis methods rely on 2D projections, we omitted the compressed gels from our size analysis to avoid misrepresenting the actual spheroid size due to a potential flattening effect.

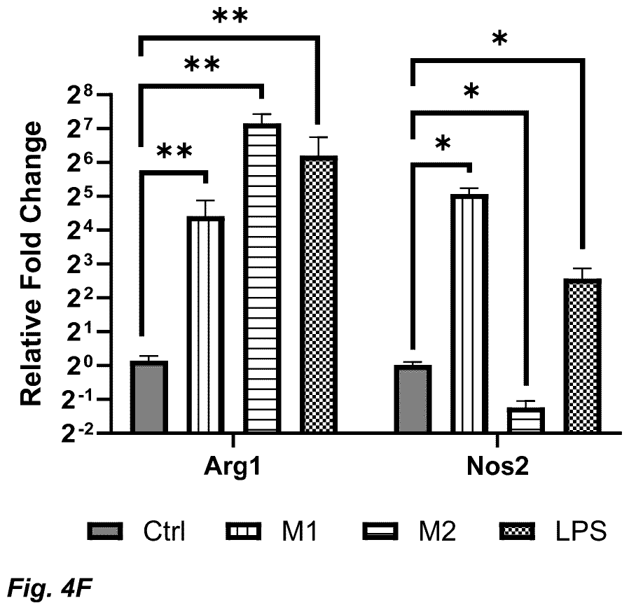

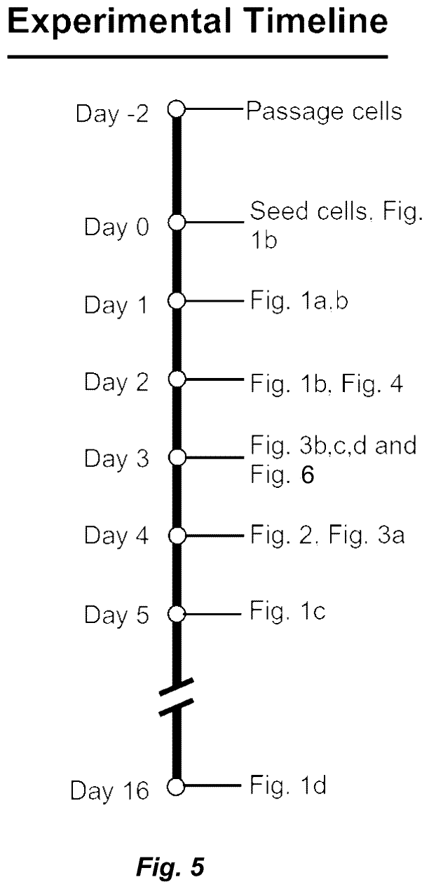

Depending on initial cell density, these gels could be maintained for several days to several weeks before they became overgrown. Different seeding densities and culture times (FIG. 5) were chosen based on requirements for cell number spheroid size, and biological material needed for each assay.

Viability and metabolic activity assays. For live/dead staining, the gels were incubated with 2 μg/ml Calcein-AM (BioLegend, 425201) and 1 μg/ml Propidium Iodide (PI, MP Biomedicals, 0219545810) in complete medium for 30 minutes at 37° C. 0.5%, 1%, and 2% agarose gels (100,000 cells/ml starting density on day 5 or else 1,000 cells/ml at day 16) were imaged on a point-scanning confocal microscope (Nikon AXR) to obtain representative spheroid images. Representative images were shown as a maximum-intensity projection of an image stack. For the viability versus agarose concentration analysis, cell-laden gels were created in a 96-well plate in 0.5%-2% agarose at an initial seeding density of 10,000 cells/ml so that individual cells could be easily distinguished. After one day in culture, three wells from each condition were imaged one day after seeding at 4× magnification in stacks with 100μm step size and 500 μm total depth (Nikon Ti2). Each stack was compressed into a maximum intensity Z projection and processed in ImageJ. For both the red and green channels, the background was subtracted using the Subtract Background function with a radius of 500 pixels. The intensity for the green and red channels was set as a uniform threshold across the image dataset, and the Analyze Particles function was used to count the number of green (live) and red (dead) cells. The viability is calculated as the number of live cells divided by the total (live plus dead) cells.

For the metabolic (3-(4, 5-dimethylthiazolyl-2)-2,5-diphenyltetrazolium bromide (MTT) assay, 50,000 cells were seeded in 50 μl of agarose, with 50 μl of media added on top. At each time point, the 100 μl of MTT reagent (ATCC, 30-1010K) was added to each well and incubated for 2.5 hours. The detergent reagent was added and incubated overnight to ensure complete dissolution of the agarose gel. The plate was read on a Spark multimode plate reader (Tecan) at 570 nm with a reference wavelength of 670 nm. Blank values for each agarose concentration were obtained from cell-free gels and subtracted from the readings to obtain the final absorbance values. Whole gel spheroid imaging. Gels were seeded with 100,000 cells/ml and cultured for three days under the various conditions described above. Representative images (5-9) of each condition were taken at 10× magnification using a phase-contrast microscope, resulting in between 26 and 87 spheroids measured per condition (Leica DMi1). Spheroid outlines were manually created for each image, and the area of identified spheroids was quantified using ImageJ's Analyze Particles function (Kumri et al., International Journal of Engineering Research and V4(11) DOI: 10.17577/IJERTV4IS110211) For representative images, images were cropped, converted to black and white, and the sharpness was set to 100% in Microsoft PowerPoint.

For the red actin-stained spheroids, 100,000 cells/ml gels at day 4 were fixed overnight in 4% paraformaldehyde in PBS (Thermo Fisher, J19943.K2) at 4° C., then rinsed with PBS and incubated with CellMask™ Orange Actin Tracking Stain (1:1000, Thermo Fisher, A57244) for 48 hours at 4° C. Representative image stacks were acquired using a multiphoton microscope (Leica Stellaris 8 DIVE).

PCR. Gels intended for PCR were seeded at a density of 10,000,000 cells/ml to ensure that enough RNA could be extracted from a small volume of agarose that was compatible with the extraction kit. RNA was isolated after 2 days of culture to avoid overgrowth and disparity in RNA quantity between conditions which encourage differential proliferation rates. For each sample, a small piece of gel (˜100 μl volume) was dissolved in 400 μl TRI-reagent (Zymo Research, R2050-1-200) and the RNA was purified using an RNA isolation kit (Zymo Research, R2051). Gene expression was analyzed using TaqMan primers for Arg1 (Thermo Fisher Scientific, Mm00475988_m1), Nos2 (Mm00440502_m1), Piezo1 (Mm01241549_m1), TRPV4 (Mm00499025_m1), Ki67 (Mm01278617_m1), Caspase 3 (Mm01195085_m1), and GAPDH (Mm99999915_g1). Gene expression was normalized to GAPDH reported as 2^{-ΔΔCt}. Each condition and gene had between 3 and 6 biological replicates. Data points that were clear outliers and that were determined by the Grubbs outlier test to be outliers were removed.

Unconfined compression testing. The compressive moduli of the agarose gels were obtained using an ElectroForce 5500 (TA Instruments) mechanical testing instrument fitted with a 5N load cell for unconfined compression testing. Low melting point agarose gels were made as described above and cast into 2 mm thick disks. Using a 6 mm diameter biopsy punch, smaller cylindrical samples were created. These were equilibrated at 37° C. in media in the same incubator used for cell culture for the duration indicated. After being initially aligned with the gel, the upper platen of a 5N load cell was programmed to descend at a rate of 0.0075 mm/s for a total of 1 mm (50%) deformation. This method was used to test three to six gels of 0.5%, 1%, and 2% agarose. The Young's modulus of each sample was determined by finding the slope of the linear region of the stress-strain curve. Data from each sample was cropped to include only the approximately linear region and an average value from 3 or 4 samples was taken to be the Young's modulus at each agarose concentration. Also see Burchett et al., bioRxiv 2025.05.13.653793; doi: https://doi.org/10.1101/2025.05.13.653793.

Computational modeling. To investigate the distribution of solid stress around the spheroids, a computational modeling approach was employed using COMSOL Multiphysics (Rathje et al., Proceedings of the National Academy of Sciences, 111, pp. 1515-1520, 2014; Fallqvist et al., Journal of the Mechanical Behavior of Biomedical Materials, Vol 59, pp. 168-184, 2016; Osiecki et al., PLOS One. 2018 Aug. 27; 13(8):e0202079). Agarose gels with concentrations of 0.5%, 1%, and 2% were modeled as linear elastic materials (E=2000, 19830, and 99596 Pa respectively) (Normand et al., Biomacromolecules 1:730-738. doi. org/10.1021/bm005583j). The spheroid geometry was obtained through image processing techniques and subsequently implemented in COMSOL. The initial geometry of the spheroid was considered as a sphere with a 5-micrometer radius. Through the application of prescribed displacement boundary conditions, the spheroids were expanded to their final geometry within the agarose gel. This approach allowed for the exploration of the solid stress and displacement distribution around the spheroids. The modeling simulations were conducted in 2-D, providing a comprehensive analysis of the mechanical stress distribution within the agarose gel environment. This methodological approach enables a detailed examination of the impact of agarose gel concentration on the mechanical behavior of macrophage spheroids in a controlled and reproducible manner.

Statistical Analysis. Statistical analyses and data visualization were done using GraphPad Prism and Excel. For cell viability, each comparison was made using the Mann-Whitney test. For MTT data, comparisons were made using a 2-way analysis of variance (ANOVA). For spheroid size, groups were compared using the Kruskal-Wallace one-way ANOVA, followed by Dunn's multiple comparisons test. PCR data was compared using the Mann-Whitney test. Error bars represent standard error of the mean (SEM) and asterisks indicate statistical significance (*p<0.05, **p<0.01, ***p<0.001, ****p<0.0001).

All publications, patents, and patent documents cited herein are incorporated by reference as though individually incorporated by reference. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. A method of testing a stimulus on an organoid comprising:
subjecting an organoid embedded in a hydrogel to a stimulus, wherein the organoid comprises a first population of live myeloid cells; and
measuring an immunological and/or physical state of the organoid to the stimulus as compared to an immunological and/or physical state of the organoid prior to contact with the stimulus or in the absence of the stimulus, wherein the stimulus comprises one or more of:
a) a compressive force;
b) one or more compounds of interest; and
c) microgravity.

2. The method of claim 1, wherein the first population of live myeloid cells comprise at least one of a monocyte, a granulocyte, an erythrocyte, and a megakaryocyte.

3. The method of claim 2, wherein the monocyte is a macrophage.

4. The method of claim 1, further comprising a second population of cells comprising one or more of adipocytes, blood cells, bone marrow cells, cardiac cells, chondrocytes, dendritic cells, endothelial cells, eosinophils, epithelial cells, fibroblasts, glial cells, glucagon-producing alpha cells, hair cells, hepatocytes, insulin-producing beta cells, keratinocytes, Langerhans cells, B and T lymphocytes, macrophages, mast cells, melanocytes, microvascular cells, monocytes, muscle cells, neurons, neutrophils, pancreatic polypeptide-producing cells, pancreatic ductal cells, pancreatic cells, parathyroid cells, pericytes (Rouget cells), pigment cells, plasma cells, pituitary cells, reticular cells, rod cells, skeletal cells, somatostatin-producing delta cells, stromal cells, and thyroid cells.

5. The method of claim 4, wherein the immunological and/or physical response comprises one or more of an increase in organoid size, a decrease in organoid size, an increase in a number of cells of the second population of cells, a decrease in a number of cells of the second population of cells, an increase in one or more cellular markers of a cell of the second population of cells, and a decrease in one or more cellular markers of cells of the second population of cells.

6. The method of claim 1, further comprising a second population of cells derived from a subject having a disease or condition resulting in an accumulation of macrophages, wherein the disease or condition comprises one or more of atherosclerosis, atherosclerotic plaques, Chronic Obstructive Pulmonary Disease (COPD), Covid-19, Hashimoto's Thyroiditis, Hepatic Fibrosis, hypertrophic scarring, an infectious disease, inflammatory phase of fatty liver disease (NASH), an intestinal disease, a neurodegenerative disease, obesity, psoriasis, rheumatoid arthritis (RA), Systemic Sclerosis systemic lupus erythematosus (SLE), Type I Diabetes, and tumors.

7. The method of claim 6, wherein the immunological and/or physical response comprises one or more of an increase in organoid size, a decrease in organoid size, an increase in a number of cells of the second population of cells, a decrease in a number of cells of the second population of cells, an increase in one or more cellular markers of a cell of the second population of cells, and a decrease in one or more cellular markers of cells of the second population of cells.

8. The method of claim 1, wherein the immunological and/or physical response comprises one or more of an increase in organoid size, a decrease in organoid size, an increase in a number of cells of the first population of live myeloid cells, a decrease in a number of cells of the population of live myeloid cells, an increase in one or more cellular markers of cells of the population of live myeloid cells, and a decrease in one or more cellular markers of cells of the population of live myeloid cells.

9. The method of claim 1, wherein the hydrogel comprises agarose in an amount of about 0.1% to about 10% of the total weight of the hydrogel.

10. The method of claim 9, wherein the hydrogel further comprises gelatin present in an amount of about 0.1% to about 10% of the total weight of the hydrogel.

11. The method of claim 1, wherein the immunological and/or physical response is measured using one or more of immunohistochemical staining, quantitative polymerase chain reaction, Fluorescence-Activated Cell Sorting, gene panel assays, fluorescence microscopy, and metabolic assays.

12. The method of claim 1, wherein the stimulus comprises the compressive force, and the measuring an immunological and/or physical response of the organoid comprises determining an amount of solid stress on the organoid based on a degree of deformation of the hydrogel surrounding the organoid and at least one mechanical property of the hydrogel.

13. An organoid comprising a first population of live myeloid cells, and optionally, at least a second population of live cells that are not myeloid cells, wherein the organoid is embedded within a hydrogel comprising agarose, and optionally, gelatin.

14. The organoid of claim 13, wherein the live myeloid cells comprise live macrophage cells.

15. The organoid of claim 13, wherein the agarose comprises about 0.5% to about 5% of total weight of the hydrogel.

16. The organoid of claim 13, wherein the hydrogel comprises a Young's moduli of about 0.5 kPa to about 150 kPa.

17. The organoid of claim 13, wherein the gelatin is present in the hydrogel in an amount of about 0.5% to about 5% of the total weight of the hydrogel.
